(19) United States
(12) Patent Application Publication
(10) Pub. No.: US 2005/0089850 A1
(43) Pub. Date: Apr. 28, 2005

(54) Genotyping by liquid chromatographic analysis of short nucleic acid fragments

(76) Inventors: Jeffrey Van Ness, Claremont, CA (US); David J Galas, Claremont, CA (US); Lori K Garrison, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/398,005

(22) PCT Filed: Oct. 1, 2001
(86) PCT No.: PCT/US01/30828

Related U.S. Application Data
(60) Provisional application No. 60/237,409, filed on Oct. 2, 2000.

(51) Int. Cl. C12Q 1/68; C07H 21/04

(52) U.S. Cl. 435/6; 536/25.4

(57) ABSTRACT

The present invention provides genotyping analysis by liquid chromatographic analysis of short nucleic acid fragments. The nucleic acid fragments are amplification products using specifically designed oligonucleotides as primers and target nucleic acids containing nucleotides of interest as templates. The oligonucleotides contain recognition sequences for restriction endonucleases that cleave outside the recognition sequences. The short nucleic acid fragments can be rapidly and reliably analyzed using liquid chromatography, optionally followed by mass spectrometry, and the nucleotides of interest identified.

### BACKGROUND OF THE INVENTION

1. Field of the Invention

The invention is in the field of molecular biology, and is more specifically directed to genotyping methods and compositions useful therein.

2. Description of the Related Art

The chromosomal mapping and nucleic acid sequencing of each of the 80,000 to 100,000 human genes, achieved through the Human Genome Project, provides an opportunity for a comprehensive approach to the identification of nucleotide loci responsible for genetic disease. Many of the 150-200 common genetic diseases and ˜600-800 of the rarer genetic diseases are associated with one or more defective genes. Of these, more than 200 human diseases are known to be caused by a defect in a single gene, often resulting in a change of a single amino acid residue. (Olsen, “Biotechnology: An Industry Comes of Age” (National Academic Press, 1986)).

Mutations occurring in somatic cells may induce disease if the mutations affect genes involved in cellular division control, resulting in, for example, tumor formation. In the germline, loss-of-function mutations in many genes can give rise to a detectable phenotype in humans. The number of cell generations in the germline, from one gamete to a gamete in an offspring, may be around 20-fold greater in the male germline than in the female. In the female, an egg is formed after a second meiotic division and lasts for 40 years. Therefore the incidence of different types of germline mutations and chromosomal aberrations depends on the parent of origin.

A majority of mutations, germline or somatic, are of little consequence to the organism since most of the genome appears to lack coding function (about 94%). Even within exon regions there is some tolerance to mutations both due to the degeneracy of the genetic code and because the amino acid substitutions may have only a slight influence on a protein's function. (See, e.g., Strong et al., N. Engl. J. Med. 325:1597 (1991)). With the development of increasingly efficient methods to detect mutations in large DNA segments, the need to predict the functional consequences (e.g., the clinical phenotype) of a mutation becomes of greater interest.

While point mutations predominate among mutations in the human genome, individual genes may exhibit peculiar patterns of mutations and, accordingly, pose different diagnostic problems. In approximately 60% of cases of Duchenne muscular dystrophy, the mutation involves a deletion of a large segment of the gigantic dystrophine gene. The elucidated mutation causing the fragile X syndrome is characterized by an increased copy number of a particular repeated sequence (CCG)ₙ. Hereditarily unstable DNA of this type may prove to be a more general phenomenon in human disease than is generally recognized.

Molecular genetic techniques have not been employed to a significant extent in the diagnosis of chromosomal aberrations in genetic and malignant disease; cytogenetics remains the preferred technique to investigate these important genetic mechanisms. In an individual with one mutated copy of a tumor suppressor gene, the remaining normal allele may be replaced by a second copy of the mutant allele in one cell per 10³-10⁴. Mechanisms causing this replacement include chromosomal nondisjunction, mitotic recombination, and gene conversion. In contrast, independent mutations destroying the function of the remaining gene copy are estimated to occur in one cell out of 10⁶.

Sensitive mutation detection techniques offer extraordinary possibilities for mutation screening. For example, analyses may be performed even before the implantation of a fertilized egg. (Holding et al., Landcet 3:532 (1989)). Increasingly efficient genetic tests may also permit screening for oncogenic mutations in cells exfoliated from the respiratory tract or the bladder in connection with health checkups. (Sidransky et al., Science 252:706, 1991). Alternatively, when an unknown gene causes a genetic disease, methods to monitor DNA sequence variants are useful to study the inheritance of the disease through genetic linkage analysis. Notwithstanding these unique applications for the detection of mutations in individual genes, the existing methodology for achieving such applications continues to pose technological and economic challenges. While several different approaches have been pursued, none are sufficiently efficient and cost effective for wide scale application.

Conventional methods for detecting mutations at defined nucleotide loci involve time-consuming linkage analyses within families using limited sets of genetic markers that are difficult to “readout.” Such methods include, e.g., DNA marker haplotyping (that identifies chromosomes with an affected gene) as well as methods for detecting major rearrangements such as large deletions, duplications, and translocations, as well as detecting single base pair mutations. These methods include scanning, screening and fluorescence resonance energy transfer (FRET)-based techniques. (See, Cotton, “Mutation Detection” (Oxford University Press, 1997)).

Highly sensitive assays that detect low abundance mutations rely on PCR to amplify the target sequence. Non-selective PCR strategies, however, amplify both mutant and wild-type alleles with approximately equal efficiency. Accordingly, low abundance mutant alleles are represented in only a small fraction of the final product. Thus, if the mutant sequence comprises <25% of the amplified product, it is unlikely that DNA sequencing approaches will be able to detect its presence. Although it is possible to quantify low abundance mutations by first separating the PCR products by cloning and subsequent probing of the clones with allele-specific oligonucleotides (ASOs), this approach is both labor intensive (requiring multiple lengthy procedures) and costly. (Saiki et al., Nature 324:163-166 (1986); Sidransky et al., Science 256:102-105 (1992); and Brennan et al., N. Engl. J. Med. 332:429-435 (1995)).

In contrast to the above, allele-specific PCR methods can rapidly and preferentially amplify mutant alleles. For example, multiple mismatch primers have been used to detect H-ras mutations at a sensitivity of one mutant in 10⁵ wild-type alleles and sensitivity as high as one mutant in 10⁶ wild-type alleles have been reported. (Haliassos et al., Nucleic Acids Res. 17:8093-8099 (1989); and Chen et al., Anal. Biochem. 244:191-194 (1997)). These successes are, however, limited to allele-specific primers discriminating through 3′ purine-purine mismatches. For the more common transition mutations, the discriminating mismatch on the 3′ primer end (i.e., G:T or C:A mismatch) will be removed in a small fraction of products by polymerase error during extension from the opposite primer on wild-type DNA. Thereafter, these error products are efficiently amplified and generate false positive signals.

It has been suggested that one means to eliminate the polymerase error problem is to deplete wild-type DNA early in the amplification cycles. Several reports have explored selective removal of wild-type DNA by restriction endonuclease digestion in order to enrich for low abundance mutant sequences. These restriction fragment length polymorphism (RFLP) methods detect approximately one mutant in 10⁶ wild-type or better. One approach has employed digestion of genomic DNA followed by PCR amplification of the uncut fragments (RFLP-PCR) to detect very low level mutations within restriction sites in the H-ras and p53 genes. (Sandy et al., Proc. Natl. Acad. Sci. USA 89:890-894 (1992) and Pourzand et al., Mutat. Res. 288:113-121 (1993)). Similar results have been obtained by digestion following PCR and subsequent amplification of the un-cleaved DNA now enriched for mutant alleles (PCR-RFLP). (Kumar et al., Oncogene 3:647-651 (1988); Kumar et al., Oncogene Res. 4:235-241 (1989); and Jacobson et al., Oncogene 9:553-563 (1994)).

Although sensitive and rapid, RFLP detection methods are limited by the requirement that the location of the mutations must coincide with restriction endonuclease recognition sequences. To circumvent this limitation, primers that introduce a restriction site (part of the recognition sequence is in the template DNA) have been employed in “primer-mediated RFLP.” (Jacobson et al., PCR Methods Applicant. 1:299 (1992); Chen et al., Anal. Biochem. 195:51-56 (1991); Di Giuseppe et al., Am. J. Pathol. 144:889-895 (1994); Kahn et al., Oncogene 6:1079-1083 (1991); Levi et al., Cancer Res. 51:3497-3502 (1991); and Mitsudomi et al., Oncogene 6:1353-1362 (1991)). Subsequent investigators have demonstrated, however, that errors are produced at the very next base by polymerase extension from primers having 3′ natural base mismatches. (Hattori et al., Biochem. Biophys. Res. Commun. 202:757-763 (1994); O'Dell et al., Genome Res. 6:558-568 (1996); and Hodanova et al., J. Inherit. Metab. Dis. 20:611-612 (1997)). Such templates fail to cleave during restriction digestion and amplify as false positives that are indistinguishable from the positive products extended from mutant templates.

Use of nucleotide analogs may reduce errors resulting from polymerase extension and improve base conversion fidelity. Nucleotide analogs that are designed to base pair with more than one of the four natural bases are termed “convertides.” Base incorporation opposite different convertides has been tested. (Hoops et al., Nucleic Acids Res. 25:4866-4871 (1997)). For each analog, PCR products were generated using Taq DNA polymerase and primers containing an internal nucleotide analog. The products generated showed a characteristic distribution of the four bases incorporated opposite the analogs.

Due, in part, to the shortcomings in the existing methodology for detecting genetic mutations, there exists an unmet need for rapid and sensitive methods for detecting mutations at defined nucleotide loci within target nucleic acids. The present invention fulfills this and other related needs by providing methods for the detection of mutations at defined nucleotide loci in target nucleic acids that, inter alia, display increased speed, convenience and specificity.

### BRIEF SUMMARY OF THE INVENTION

As disclosed in detail herein below, methods according to the present invention are based on digestion of target nucleic acids with restriction endonucleases that cleave outside their recognition sites, thus producing short nucleic acid fragments, and subsequently the successful use of liquid chromatography in characterizing such short fragments.

In one aspect, the present invention provides methods for identifying one or more nucleotide(s) at a defined location in a double-stranded target nucleic acid, comprising the following steps:
(a) forming a mixture of the target nucleic acid, a first oligonucleotide primer (ODNP) and a second ODNP,
   wherein each of the first and the second ODNPs comprises a 5′ end and a 3′ end, a first portion of each ODNP at the 5′ end and a second portion of each ODNP at the 3′ end are at least substantially complementary to a first portion and a second portion of the target nucleic acid, and 4-8 nucleotides between the first portion and the second portion of the ODNP comprise a recognition site for a restriction endonucleotide (RE) that cleaves outside its recognition site, and
   wherein each ODNP is complementary to an opposite strand of the target nucleic acid, the first and the second ODNPs are complementary to two non-contiguous regions of the target nucleic acid, and the defined position is between the two non-contiguous regions;
(b) amplifying the target nucleic acid using the first and the second ODNPs;
(c) digesting the amplification product of step (b) with the restriction endonuclease(s) that recognize the recognition sequences in the first and the second ODNPs; and
(d) characterizing a short digestion product of step (c) with liquid chromatography.

In some embodiment, the first portion of each ODNP at the 5′ end and the second portion of each ODNP at the 3′ end are exactly complementary to the first portion and the second portion of the target nucleic acid.

In certain embodiments, the first portion of each ODNP is at least 6, 8, 10, 12 nucleotides in length. In some embodiments, the second portion of each ODNP is at least 1, 3, 5 or 7 nucleotides in length. Preferably, the second portion of each ODNP is at most 16, 18, 20, 22 or 24 nucleotides in length.

In certain embodiments, the distance between the first and the second portions in the target nucleic acid is up to 10 nucleotides in length. Preferably, the distance is 4-8 nucleotides in length. Also preferably, the target nucleic acid does not comprise a recognition site between the first and the second portions that is recognizable by a restriction endonuclease cleaving outside its recognition site.

In some embodiments, the recognition sequences in the first and the second ODNPs are the same. In other embodiments, the recognition sequences in the first and the second ODNPs are different.

In some embodiments, the RE(s) that recognizes the recognition sequences in the first and the second ODNPs are Type IIS RE(s). In a preferred embodiment, the RE is Bpm I.

In certain embodiments, the distance between the two non-contiguous regions in the target nucleic acid is 1 to 30, preferably 1 to 20, more preferably 1-10 nucleotides in length.

In some embodiments, the target nucleic acid is genomic DNA or cDNA. In certain embodiments, the nucleotide(s) at the defined location is associated with a disease. In other embodiments, the nucleotide(s) at the defined location is associated with drug resistance of a pathogenic microorganism.

In other aspects, the invention provides a method of performing chromatography in order to characterize and identify nucleic acid molecules, particularly nucleic acid molecules formed from less than 20 nucleotides. Thus, in one aspect, the present invention provides a method of performing liquid chromatography comprising: applying two nucleic acid molecules to a liquid chromatography column, where the two nucleic acid molecules have an identical number of nucleotide bases, but have different nucleotide sequences; and eluting the two nucleic acid molecules from the column with an elution buffer so that the two nucleic acid molecules have different elution times; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid. Optionally, the two nucleic acid molecules are composed of identical nucleotides, but the order of the nucleotides in the two nucleic acid molecules is non-identical. As another option, the two nucleic acid molecules each have the sequence 5′-n-X-m⁻³′, where n and m each represent a sequence of from 0-10 nucleotides, and X represents a single nucleotide, and the two nucleic acid molecules each have the same sequences n and m, but differ in the identify of the nucleotide at location X.

In another aspect, the present invention provides a method method of performing liquid chromatography comprising: applying two nucleic acid molecules to a liquid chromatography column, where the two nucleic acid molecules have an identical number of nucleotide bases within the range of 2-10, but have different nucleotide sequences, and the liquid chromatography column is a reverse phase chromatography column; and eluting the two nucleic acid molecules from the column with an elution buffer so that the two nucleic acid molecules have different elution times; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid.

The present invention also provides a method of performing liquid chromatography comprising: applying two nucleic acid molecules to a liquid chromatography column, where the two nucleic acid molecules have an identical number of nucleotide bases, but have different nucleotide sequences; and eluting the two nucleic acid molecules from the column with an elution buffer so that the two nucleic acid molecules have different elution times; the elution buffer being formed from Buffer A and Buffer B, where Buffer A comprises water and an ammonium salt that is formed from a secondary or tertiary amine complexed with an organic or inorganic acid; and Buffer B comprises water, organic solvent, and an ammonium salt that is formed from a secondary or tertiary amine complexed with an organic or inorganic acid; where elution buffer of incrementally increasing organic solvent concentration is applied to the column.

In another aspect, the present invention provides a method of performing liquid chromatography and mass spectrometric analysis comprising: applying a plurality of pairs of nucleic acid molecules to a liquid chromatography column, where each pair of nucleic acid molecules is formed from two nucleic acid molecules that have an identical number of nucleotide bases, but have different nucleotide sequences; eluting the plurality of pairs of nucleic acid molecules from the column with an elution buffer so that the two nucleic acid molecules that form each pair have different elution times; and characterizing each nucleic acid molecule by mass spectroscopy; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid.

The present invention also provides a composition referred to herein as Buffer B comprising water, the reaction product of secondary or tertiary amine with organic or inorganic acid, and organic solvent. In another aspect, the present invention provides a composition comprising water, the reaction product of secondary or tertiary amine with organic or inorganic acid, organic solvent, and two nucleic acid molecules, where the two nucleic acid molecules have an identical number of nucleotide bases, but have different nucleotide sequences.

The present invention also provides a kit for chromatographic analysis comprising: (a) a container holding components comprising water and the reaction product of secondary or tertiary amine with organic or inorganic acid (Buffer A); and (b) a container holding components comprising the components of (a) and organic solvent (Buffer B).

These and other aspects of the present invention are described in further detail below.

### BRIEF DESCRIPTION OF THE SEVERAL VIEWS OF THE DRAWING(S)

FIG. 1 is a diagram of a double-stranded target nucleic acid.

FIG. 2 is a diagram showing the relationship between the first ODNP and the top strand of the target nucleic acid.

FIG. 3 is a diagram showing the situation where the first and second portions of a first ODNP are complementary to bases of the top strand of a target nucleic acid so that there are more nucleotides separating the first and second portions than there are nucleotides separating the corresponding complementary regions in the top strand.

FIG. 4 is a diagram of major steps in one aspect of the present method for identifying a nucleotide at a defined position in a target nucleic acid using an ODNP pair and an exemplary restriction endonuclease recognition sequence for Fok I.

FIG. 5 is a high pressure liquid chromatogram (HPLC) of a set of 4, 6, 8 and 10 nucleotide ODNP.

FIGS. 6A and 6B show HPLC separation of three 8-mers (FIG. 6A) and three 10-mers (FIG. 6B).

FIGS. 7A and 7B show the HPLC separation of one 4-mer, one 6-mer, three 8-mers and three 10-mers (FIG. 7A) and the elution of two 6-mers (FIG. 7B).

FIG. 8 is a control HPLC chromatogram.

FIG. 9 shows HPLC fractionation and detection of short fragments generated by the Fok I double digestion.

### DETAILED DESCRIPTION OF THE INVENTION

The present invention provides methods of obtaining genetic information such as genotype analysis to identify nucleic acid variations. In one aspect the invention it prepares short nucleic acid segments by amplifying a target nucleic acid using specially designed oligonucleotide primers (ODNPs). The amplification product is subsequently digested with restriction endonuclease(s) that recognize restriction endonuclease recognition sequence(s) that have been incorporated into the product via the specially designed ODNPs. The digestion product includes one or more short polynucleotide segments that can be analyzed by liquid chromatography to identify whether nucleic acid variation is present in the target nucleic acid. This method is simple to implement (e.g., no purification of digestion products required before liquid chromatographic analysis), accurate, and more economical than genotyping methods using mass spectrometric analysis (Laken et al., published PCT application No. WO 00/31300). The present method can be used in a variety of applications such as genetic analysis for hereditary diseases, tumor diagnosis, disease predisposition, forensics or paternity, crop cultivation, animal breeding, expression profiling of cell function and/or disease marker genes, and identification and/or characterization of infectious organisms that cause infectious diseases in plants or animals and/or that are related to food safety.

The target nucleic acid can be any polynucleotide that contains an (one or more) unknown nucleotide at a defined location. In one aspect, the target nucleic acid is formed by an organism, and has been at least partially, preferably completely, isolated from the organism. Methods by which the target may be isolated from the organism are well known and often practiced in the art. The subject organism can be any organism, for example, a human or other animal, a plant, a fungus, or a microorganism such as a bacterium or a virus. In another aspect the target nucleic acid is synthetic in origin, i.e., has been made according to human intervention or design. For instance, many companies are now in the business of making “genes” or other long polynucleic acids, and these materials may be the target nucleic acid of the present invention.

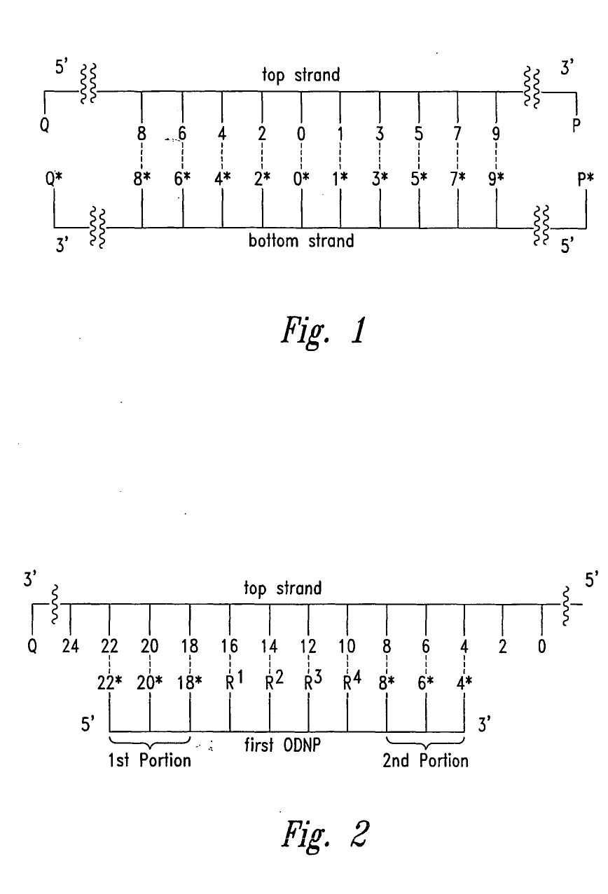

The target nucleic acid may be represented by the generalized formula shown in FIG. 1. It is composed of two strands, each strand being a nucleic acid molecule, which are arbitrarily identified and distinguished by the names “top strand” and “bottom strand.” In general, these two strands may be given other convenient names, where first/second, coding/non-coding, and 3′/5′ are other conventions used in the art to distinguish the two strands that form the double-stranded target. The top and bottom strands are each either an oligonucleotide (having up to 100 nucleotides) or a polynucleotide (having more than 100 nucleotides). Each strand is identified as having a 3′ end and a 5′ end, which is conventional naming used in the nucleic acid art for oligonucleotides and polynucleotides.

Also regarding the general formula, each of the numbers represents a base (adenine (A), guanine (G), cytosine (C) or thymine (T)) connected to a neighboring base by a sugar and a phosphate group represented by two straight lines that together form a right angle, e.g., [symbol] , [symbol] , [symbol] , and [symbol] . Either the base designated by “0” or the base designated by “0*” is the base at the “defined position” in the method of the present invention. This position is “defined” in that the investigator knows the base sequence in the 3′ direction from the defined position, i.e., the sequence 1 3 5 7 9 etc., and also knows the base sequence in the 5′ direction as measured from the defined position, i.e., the sequence 2, 4, 6, 8, etc. However, the investigator does not know the identity of the base at the defined position. The present invention provides a method by which the investigator may identify the base at the defined position.

As regards Q, Q*, P and P* in the general formula, these designations denote the highest even number (in the case of Q and Q*) and the highest odd numbers (in the case of P and P*) that will identify the number of bases in the target nucleic acid. For instance, taking the simplest example, if Q and Q* are each “10” and P and P* are each “11,” then the target nucleic acid contains twelve nucleotides in each of the top and bottom strands. In general, the upper strand contains ½Q*+½(P+1)+1 nucleotides, while the bottom strand contains ½Q*+½(P*+1)+1 nucleotides. The upper and lower strands may, but need not contain the same number of nucleotides. That is, P does not necessarily equal P*, and Q does not necessarily equal Q*.

Also in the formula, each number and its complement, e.g., 0 and 0*, 1 and 1*, 2 and 2*, etc., represent a base pair selected from A and T, and G and C. The dashed line between a number and its complement represents hydrogen bonding between the two bases.

The present invention forms a mixture that includes, among other possible components, a target nucleic acid as described above, a first oligonucleotide primer (first ODNP) and a second oligonucleotide primer (second ODNP). As described next, the first and second ODNPs must meet certain requirements vis-à-vis the target nucleic acid. Primarily, the first ODNP must be able to (1) prime an extension and/or amplification reaction in combination with the target nucleic acid; (2) be able to hybridize to the top strand of the target nucleic acid at a location 3′ from the defined position in the target nucleic acid and not overlapping the defined position, and (3) contain a sequence that will be recognized by a restriction endonuclease.

As shown in FIG. 2, the first ODNP contains a nucleotide sequence such that it will hybridize to a region of the top strand that is located in the 3′ direction from the defined location of the target nucleic acid. FIG. 2 shows that complements 4/4*, 6/6*, 8/8*, 18/18*, 20/20* and 22/22* are present between the first ODNP and the top strand of the target nucleic acid, where each of these complements is selected from A/T, T/A, G/C, and C/G. These complements provide thermal stability when the first ODNP hybridizes to the top strand. As used herein, an ODNP is “exactly complementary” to its template sequence if every nucleotide of the ODNP is complementary to every corresponding nucleotide of the template sequence wherein A is complementary to T and G is complementary to C. Noteworthy is that some mismatching of bases can occur according to the present invention even between the bases in the first and second portions of the first ODNP and the complimentary locations in the top strand. However, since the presence of mismatches destabilizes the duplex, the duplex cannot have too many mismatches or else the duplex will either not form at all or will not be sufficiently stable under the extension/amplification conditions to effectively allow the extension/amplification reaction to occur. When one or more mismatches are present between the top strand and the first and/or second regions of the first ODNP, but those mismatches do not impair the practice of the present invention, then the first ODNP and the top strand are substantially complimentary. Typically, an ODNP is substantially complementary to a target nucleic acid when it is at least 90% identical to the complement of the target over the length of the ODNP as determined by the Smith-Waterman homology search algorithm as implemented in the MPSRCH program (Oxford Molelcular) using an affine gap search with a gap open penalty of 12 and a gap extension penalty of 1.

In addition, as illustrated in FIG. 2, there is a contiguous sequence of bases within the first ODNP, denoted R¹ through R⁴, that do not completely hybridize to the top strand of the target nucleic acid. This sequence R¹-R⁴ is selected, according to the present invention, to be a recognition sequence for a restriction endonuclease. One or more of the nucleotides R¹-R⁴ may, coincidentally, hybridize to the corresponding base in the top strand of the target nucleic acid. However, it is unnecessary that any of the bases R¹-R⁴ hybridize to any base in the top strand of the target nucleic acid. Should such hybridization occur, it is not detrimental to the present invention, and may provide additional, desirable stability for the duplex formed between the first ODNP and the top strand of the target nucleic acid.

In an ODNP of the present invention, there is a sequence of nucleotides located between the recognition sequence and the 5′ end of the ODNP that will be referred to herein as the first portion of the ODNP. In addition, in an ODNP of the present invention, there is a sequence of nucleotides located between the recognition sequence and the 3′ end of the ODNP that will be referred to herein as the second portion of the ODNP. Referring to FIG. 2, which is illustrative only, the first portion consists of 18*, 20* and 22*, while the second portion consists of 8*, 6* and 4*. These first and/or second portions are the regions of the ODNP that are designed to be sufficiently complementary to the top strand of the target nucleic acid such that the first ODNP will hybridize to the top strand to a sufficient extent that the first ODNP can prime an polymerase catalyzed extension reaction whereby the first ODNP is extended in the 3′ direction to provide a partial complement of the top strand of the target nucleic acid. Noteworthy, and a necessary feature of the present invention, is that this extension reaction copies into the extended first ODNP a complement of the nucleotide at the defined position in the target nucleic acid. In this way, the base information present at the defined position of the top strand of the target nucleic acid is transferred into the extended first ODNP.

For convenience, those bases of the top strand of the target nucleic acid that are exactly complementary or substantially complementary to the first portion of the first ODNP will be referred to herein as the first portion of the top strand of the target nucleic acid. Likewise, those bases of the top strand of the target nucleic acid that are exactly complementary or substantially complementary to the second portion of the first ODNP will be referred to herein as the second portion of the top strand of the target nucleic acid.

An ODNP of the present invention must have either a first portion or a second portion, and preferably has both a first and second portion. If the ODNP does not contain a second portion, then the polymerase catalyzed extension reaction will generally be quite slow to occur because the polymerase requires a double-stranded end in order to begin the extension reaction. In the absence of this second portion, the necessary double-stranded end will only occur occasionally, that is, when the terminal base of the recognition sequence in the primer (R⁴ in FIG. 2) happens hybridize to the base opposite itself (10 in FIG. 2) in the top strand of the target nucleic acid sequence. Since R⁴ and 10 will not necessarily be complementary, this hybridization reaction between R⁴ and 10, when it occurs, will occur only fleetingly due to the high energy of the hybrid. Accordingly, it is preferred according to the present invention that the first ODNP contain a second portion, where that second portion is substantially or, preferably, exactly complementary to the top strand of the target nucleic acid, and as an additional preferred embodiment, the second portion contains at least 1, or at least 3, or at least 5, or at least 7, or at least 9 nucleotides. Preferably, the second portion is at most 16, 18, 20, 22, or 24 nucleotides in length.

If the first ODNP does not contain a first portion, then it is necessary that the first ODNP contain a second portion that is exactly or substantially complementary to the first portion of the top strand of the target. Preferably, the first ODNP contains both a first and second portion, where together these two portions stably hold the first ODNP in a hybridized arrangement with the top strand of the target nucleic acid, so that the polymerized extension reaction will readily proceed. As for the size of the first portion, this is generally in the range of 2-20 nucleotides. The first portion may contain at least 6, 8, 10, 12, or 14 nucleotides. If the second portion is relatively long, on the order of 10 nucleotides, then the first portion need not be too long, because the second portion alone will be able to hold the first ODNP in a hybridized state with the top strand of the target nucleic acid. However, if the second portion is relatively short, on the order of 2-4 nucleotides, then the first portion must be more substantial, on the order of 8-20 nucleotides.

To some extent, the total number of nucleotides in the first and second portions depends on the bases present in the first and second portions. As is well known in the art, it is empirically observed that a G/C base pair provides more stability to a hybrid than does an A/T base pair, the reason probably being that G/C base pairs are formed from three hydrogen bonds while an A/T base pair is formed from only two hydrogen bonds. In a preferred embodiment of the present invention, which generally works regardless of the identity of the bases present in the first and second portions of the first ODNP, the first ODNP has a second portion that contains 4-8 nucleotides where those nucleotides are substantially complementary to the second portion of the target nucleic acid.

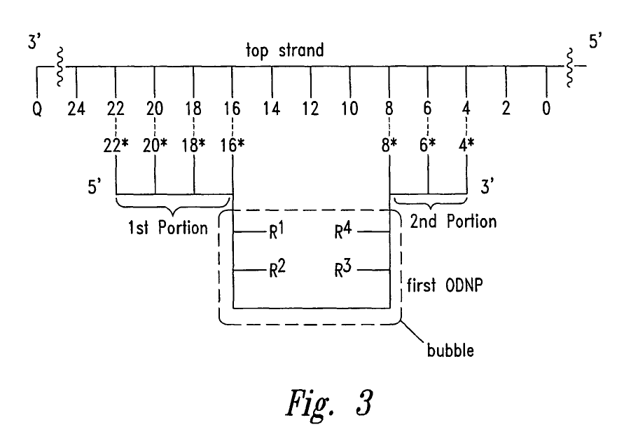

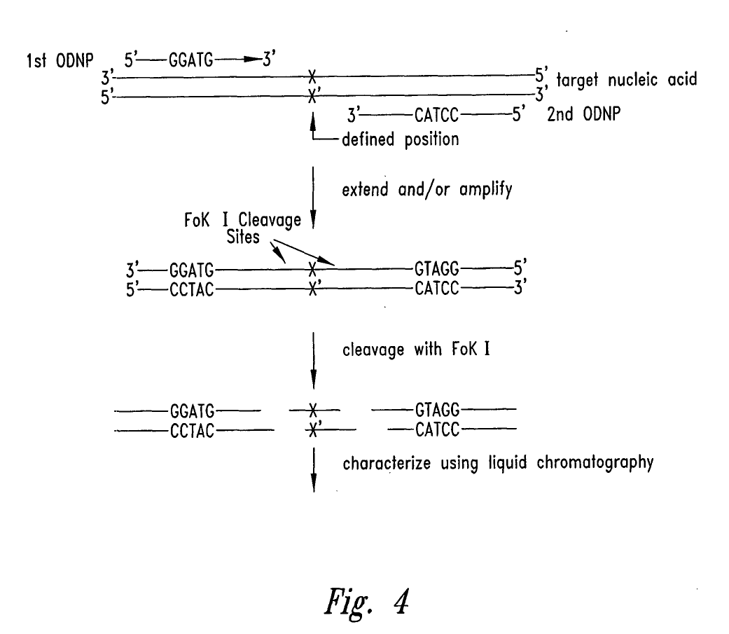

Another factor to consider when designing a first and second portion of a first ODNP is the location of the restriction site. In FIG. 2, the first and second portions are designed such that the restriction site is located across from an equal number of nucleotides in the top strand of the target nucleic acid. However, this need not be the case, as the present invention contemplates that the number of nucleotides separating the first and second portions of the first ODNP may be less than, equal to, or greater than the number of nucleotides that separate the first and second portions of the top strand of the target nucleic acid. The situation where there are more nucleotides separating the first and second portions of the first ODNP than there are nucleotides separating the first and second portions of the top strand of the target nucleic acid is illustrated in FIG. 3. The situation where there are less nucleotides separating the first and second portions of the first ODNP than there are nucleotides separating the first and second portions of the top strand of the target nucleic acid would have a complementary appearance.

FIG. 3 illustrates the situation where the first and second portions of the first ODNP are complementary to bases of the top strand of the target nucleic acid such that there are more nucleotides separating the first and second portions than there are nucleotides separating the corresponding complementary regions in the top strand. In this case, the restriction enzyme recognition sequence will need to form a “bubble” as identified in this Figure.

If the first ODNP is designed such that a bubble will be formed when the first ODNP hybridizes to the top strand of the target nucleic acid, it is possible according to the present invention that the bubble will contain nucleotides that are not part of the restriction enzyme recognition sequence. Another possibility is that one or more of the nucleotides present in the restriction enzyme recognition sequence will be able to hybridize to the top strand of the target nucleic acid, and accordingly even when a bubble is formed, some of the nucleotides that form the restriction enzyme recognition sequence may not be part of the bubble. Likewise, even if the first and second portions of the first ODNP are designed such that there is not a bubble formed when the first ODNP hybridizes to the top strand of the target nucleic acid, the nucleotides interposed between the first and second portions of the first ODNP need not all be part of a recognition sequence for a restriction enzyme. Generally, however, the first and second portions of the first ODNP are separated by 0-16, preferably 4-8 nucleotides, where these nucleotides contain a restriction enzyme recognition sequence.

Another point to consider when designing the first ODNP is that the first ODNP need not hybridize to the nucleotide adjacent to the defined position in the target nucleic acid. This is illustrated in FIG. 2, where the first ODNP actually hybridizes to the top strand of the target nucleic acid such that there is a gap of a single nucleotide (denoted “2”) between the second portion of the top strand of the target nucleic acid and the defined position of the target nucleic acid. According to the present invention, this “gap” ranges in length from 0 nucleotide (i.e., no gap is present) to not more than 20, preferably not more than 10, nucleotides. While the invention may be practiced with a gap greater than 20 nucleotides, this is typically not advantageous because the inventive method then affords a relatively large digestion product when restriction enzyme acts on amplification product of first and second ODNPs and the target nucleic acid. Typically, greater discrimination is afforded according to the present invention when the digestion product is shorter, and thus introducing a larger gap when designing first (and second) ODNPs is not typically advantageous. Thus, in one preferred aspect of the present invention, there is no gap between the first portion of the ODNP, while in another aspect the gap is only a single nucleotide.

The guidelines provided above for designing a first ODNP to hybridize to the top strand of the target nucleic acid are the same as the guidelines that should be used in designing a second ODNP to hybridize to the bottom strand of the target nucleic acid. Accordingly, less specific discussion will be provided regarding the design of the second ODNP. It is important to note, however, that the parameters that were chosen for the first ODNP, e.g., the number of nucleotides in the first portion of the first ODNP, the number of nucleotides in the second portion of the first ODNP, the number of bases separating the first and second portions of the first ODNP, whether the second portion of the first ODNP is exactly complementary to the second portion of the top strand of the target nucleic acid, or is merely substantially complementary, whether hybridization between the first ODNP and the top strand of the target nucleic acid produces a bubble in the top strand of the target nucleic acid or in the first ODNP, etc. need not be the same between the first and second ODNPs. Thus, for example, the first ODNP may have a first portion containing 7 nucleotides, while the second ODNP may have a first portion containing 5 nucleotides.

Basically, the second ODNP is designed so that it will hybridize to the bottom strand of the target nucleic acid, at a location 3 of the defined position of the bottom strand, i.e., in the 3′ direction from the nucleotide defined as 0*. The second ODNP will hybridize to the bottom strand by way of first and/or second portions, just as the first ODNP hybridized to the top strand of the target nucleic acid by way of first and second portions. The second ODNP is designed so that the hybridization product between it and the bottom strand of the target nucleic acid may, or may not, have a gap between the double-stranded hybridization product and the defined position of the bottom strand. Importantly, while the second ODNP will contain a restriction enzyme recognition sequence, the enzyme that recognizes the sequence in the first ODNP need not be the same as the enzyme that recognizes the sequence in the second ODNP. Likewise, the restriction enzyme recognition sequence of the first ODNP need not be identical to the restriction enzyme recognition sequence of the second ODNP. In one aspect of the invention, the first and second ODNPs share the same restriction enzyme recognition sequence. Either the first ODNP, or the second ODNP, or both may comprise at least 10, 15, 20, 25, 30, 35, 40, 45, 50, 60 or 70 nucleotides.

The first and second ODNPs are at least partially complementary to two non-contiguous regions of opposing strands of a double-stranded target nucleic acid. These two regions may be separated from each other by 1 to about 10, 15, 20, 25, 30, 35, or 40 base pairs, and preferably by 1 to 20 base pairs. The region that separates these two regions contains the defined position at which a nucleotide of interest resides.

The present invention uses restriction endonuclease(s) that cleave nucleic acid molecules outside their recognition sites. A “restriction endonuclease” or “restriction enzyme” refers to the class of nucleases that bind to unique double-stranded nucleic acid sequences referred to herein as a recognition site, and generate a cleavage in the double-stranded nucleic acid that results in either blunt, double-stranded ends, or single-stranded ends with either a 5′ or a 3′ overhang. For convenience, a single-stranded nucleic acid molecule may be described herein as comprising a recognition sequence for a restriction enzyme. However, the actual recognition site for the endonuclease consists of the recognition sequence and the complement thereof. A restriction endonuclease will bind to a recognition site and then cleave each strand that forms the recognitions site. The position at which the endonuclease cuts the double-stranded nucleic acid molecule is referred to as the “cleavage site.” The position of the cleavage site is relative to the recognition site and is a characteristic of the endonuclease. The restriction endonuclease can be, for example, a Type IIS restriction endonuclease such as BpmI, BsgI, Eco57 I, or Fok I. Type IIS restriction endonucleases have asymmetric recognition sites and cleave at a specific distance of up to about 20 bp from their recognition site. Using a restriction endonuclease that cleaves outside the recognition site is advantageous because the product of endonuclease digestion can be a nucleic acid fragment smaller than that if the endonuclease cleaved within the recognition site, thereby generating a fragment that is particularly suitable for liquid chromatographic analysis. In this embodiment, the restriction endonuclease should have a cleavage site distal from its recognition site by at least 3, 4, 5, 6, 8, 10, 12, or 15 nucleotides, and preferably by at least 8 nucleotides.

ODNPs according to the invention can be synthesized by any method known in the art for oligonucleotide synthesis. For instance, solid phase oligonucleotide synthesis can be performed by sequentially linking 5′ blocked nucleotides to a nascent oligonucleotide attached to a resin, followed by oxidizing and unblocking to form phosphate diester linkages. ODNPs of the present invention may then be isolated. The term “isolated” as used herein refers to a molecule that is substantially free of undesired contaminants, such as molecules having other sequences.

To carry out genotyping according to one aspect of the invention, the ODNPs are used to amplify a segment from a sample of a target nucleic acid. The term “amplification” as used herein refers to any process using a pair of ODNPs described above that produces a target nucleic acid fragment between, and including, the portion complementary to the 5′ ends of the pair of ODNPs. Any amplification method known in the art may be used such as PCR methods disclosed in U.S. Pat. Nos. 4,683,195, 4,683,202 and 4,800,159. In addition, PCR methods are also described in several books, e.g., Gelfand et al., “PCR Protocols: A Guide to Methods and Application” (1990); Burke (ed), “PCR: Essential Techniques”; McPherson et al. “PCR (Basic: From Background to Bench).” Each of the above reference books is incorporated herein by reference in its entirety. Briefly, in PCR, two ODNPs are prepared that are complementary to regions on opposite complementary strands of the target nucleic acid. An excess of deoxynucleoside triphosphates is added to a reaction mixture containing the target nucleic acid along with a DNA polymerase (e.g., Taq or Pfu polymerase). If the target nucleic acid comprises the sequence of interest, the ODNPs will bind to the target and the polymerase will cause the ODNPs to be extended along the target nucleic acid sequence by the addition of nucleotides. By raising and lowering the temperature of the reaction mixture, the extended ODNPs will dissociate from the target to form reaction products, excess ODNPs will bind to the target and to the reaction product, and the process is repeated until the sequence of interest is amplified.

Exemplary PCR conditions according to the present invention include, but are not limited to, the following: 100 μl PCR reactions comprise 100 ng target nucleic acid; 0.5 μM of each first ODNP and second ODNP; 10 mM Tris, pH 8.3; 50 mM KCl; 1.5 mM MgCl₂; 200 μM each dNTP; 4 units Taq™ DNA Polymerase (Boehringer Mannheim; Indianapolis, Ind.), and (optionally) 880 ng TaqStart™ Antibody (Clontech, Palo Alto, Calif.). Exemplary thermocycling conditions may be as follows: 94° C. for 5 minutes to achieve initial denaturation; 45 cycles at 94° C. for 30 seconds, 60° C. for 30 seconds, 72° C. for 1 minute; final extension at 72° C. for 5 minutes. Exemplary nucleic acid polymerases may include one of the thermostable DNA polymerases that are readily available in the art such as, e.g., Taq™, Vent™ or PFU™. Depending on the particular application contemplated, it may be preferred to employ one of the nucleic acid polymerases having a defective 3′ to 5′ exonuclease activity.

The amplified product is cleaved (digested) using a restriction endonuclease whose recognition site comprises a recognition sequence present in an ODNP. When the enzyme cleaves the double-stranded amplification product, it breaks a covalent bond at a discrete location on each strand. Digestion of a double-stranded nucleic acid molecule with a restriction endonuclease refers to the process of allowing the endonuclease to bind to its recognition site, cleave at its cleavage site, and release the cleavage products (segments or fragments, these terms being used synonymously). In one aspect of the invention, each member of an ODNP pair of this invention (first and second ODNPs) contains a recognition sequence for the same restriction endonuclease, so that digestion of the amplified product with the endonuclease will result in cleavage at two sites and consequently the release of a defined fragment of the product. In another aspect, the two ODNPs of an ODNP pair contain recognition sequences for different restriction endonucleases. In other words, the first ODNP has a recognition sequence that differs from that of the second ODNP. In this later aspect, the amplification products described above are digested with two restriction endonucleases that recognize the recognition sites generated by the first and the second ODNPs. However, no matter whether the amplification product is digested with one or two restriction endonucleases that recognize the recognition sites in the ODNP-generated product, the product of the restriction endonuclease digestion will be a short fragment of double-stranded DNA, whose length can be from 1, 2, 3, 4, 5, 6, 7, 8, 9, or 10 to about 14, 16, 18, 20, 22, 24, 26, 30, 35, or 40 bp, including every combination therein.

According to the present invention, the short fragment of double-stranded nucleic acid can be denatured, and the resulting single-stranded segments can be subjected to liquid chromatography to provide a retention time upon identification in the eluent using, e.g., UV absorbance. In other words, no purification procedure is needed before the digestion products are subjected to liquid chromatographic analysis. In certain embodiments, the nucleic acid-containing eluent from liquid chromatographic analysis may be further characterized by techniques known in the art that are applicable to short oligonucleotide fragments, such as mass spectrometry. This further analysis may be used to validate the conclusion reached after liquid chromatographic analysis.

A surprising discovery of the present invention is that liquid chromatography can be used to identify small nucleic acid molecules and/or distinguish small nucleic acid molecules from one another. Remarkably, two nucleic acid molecules may be of the same length, i.e., contain the same number of nucleotides, and differ from each other only in their sequences at one nucleotide position, and yet these two molecules can be distinguished from one another based on retention time obtained from liquid chromatographic analysis performed according to the present invention. Also remarkable is that two nucleic acid molecules that are of the same length and of the same nucleotide composition, and differ from each other only in the order in which the nucleotides are arranged, may be distinguish from one another based on retention time obtained from liquid chromatographic analysis performed according to the present invention. As one example, the order of two nucleotides in a nucleic acid molecule can be switched to provide a variant nucleic acid molecule, and the original and variant nucleic acid molecules may be distinguished by their different retention times as observed by liquid chromatographic analysis (see, e.g., 8merB and 8merC in Example 1). As discussed in more detail below, an important factors in being able to use retention time to distinguish two similar nucleic acid molecules is the selection of the mobile phase for the chromatographic analysis.

The mobile phase is a gradient formed from two buffer solutions (Buffer A and Buffer B). That is, after the nucleic acid molecules have been applied to the column, Buffer A, or an elution buffer that is largely Buffer A, is used to elute the molecules. During the course of the chromatography, Buffer B is gradually (incrementally) added to Buffer A so that the eluting buffer gradually becomes enriched in the components specific to Buffer B. Buffer A is an aqueous solution of an (one or more) ammonium salt, while Buffer B is an organic solution of an (one or more) ammonium salt. As referred to herein, an aqueous solution necessarily contains water, but may also contain non-aqueous components including organic components, e.g., organic solvent(s). As referred to herein, an organic solution necessarily contains organic material, but may and typically will also contain non-organic components including inorganic components, e.g., water as a solvent. The water used in Buffers A and/or B is preferably HPLC grade water, where this quality of water is well known in the art.

The ammonium salt(s) present in Buffer A need not be identical to the ammonium salt(s) present in Buffer A, however, in one aspect of the invention Buffer A and Buffer B contain the same ammonium salts. Either of Buffer A or Buffer B may, independently of the other, contain more than one ammonium salt, however, in one aspect of the invention Buffer A and Buffer B each contain a single ammonium salt structure. In one aspect of the invention, each of Buffer A and Buffer B contain a single ammonium salt and furthermore these two buffers contain the same ammonium salt.

Referring to Buffer A, in one aspect of the invention water is the only solvent present in this buffer. In other aspects of the invention, water constitutes at least 99%, at least 97%, at least 95%, at least 90%, at least 85%, or at least 80% of the total volume of solvent present in Buffer A. Referring to Buffer B, in one aspect of the invention this buffer contains both organic solvent and water. Typically, water is necessarily present in Buffer B in order to solubilize the ammonium salt in the buffer. In various aspects of the invention, the organic solvent constitutes up to 75%, up to 60%, up to 45%, up to 30%, or up to 15% of the total volume of solvents present in Buffer B, with water contributing the residual volume of solvent. In various aspects of the invention, the volume ratio of organic solvent:water in Buffer B is 5-75:95-25, or 10-50:90-50; or 15-40:85-60; or 20-35:80-65 with the total of the organic solvent and the water equaling 100.

As to the organic solvent, this is preferably a solvent that is very miscible with water. Preferably, the organic solvent is miscible with water to an extent of at least about 5 vol % based on the total volume of water and organic solvent, and in various aspects is miscible to an extent of 10 vol %, 20 vol %, 30 vol %, 40 vol %, or 50 vol % (i.e., an equal volume of water and organic solvent forms a homogeneous solution). As referred to herein, a solvent is “organic” so long as it contains at least one carbon. The organic solvent is preferably a liquid at room (ambient, standard) temperature and pressure. Such solvents are very well known in the art. In one aspect, the organic solvent is an alcohol, ie., an organic liquid at room temperature than contains at least one hydroxyl (OH) group. Exemplary alcohols include, without limitation, methanol, ethanol, ethylene glycol, iso-propanol, n-propanol, propylene glycol, and glycerol. Typically, as the number of carbon atoms present in the alcohol increases, the alcohol becomes less soluble in water, and the alcohol must contain more than one hydroxyl group in order to embody effective miscibility with water. In another aspect the organic solvent is acetonitrile. In yet another aspect the organic solvent is dimethylsulfoxide (DMSO).

As to the ammonium salt(s), these are selected from primary (RNH₂), secondary (R₂NH) or tertiary (R₃N) amines that are complexed with protic acid (H-A). In one aspect, the ammonium salt is a primary amine, in another aspect the ammonium salt is a secondary amine, in yet another aspect the ammonium salt is a tertiary amine. In another aspect the ammonium salt is selected from secondary and tertiary amine salts. The ammonium salt should be soluble in the solvent(s) that form the buffer into which the ammonium salt is to be added. In various aspects the ammonium salt is water soluble at a concentration up to 1,000 mM, or up to 800 mM, or up to 600 mM, or up to 400 mM, or up to 200 mM, or up to 100 mM. Particularly if the liquid chromatographic analysis will be followed by mass spectroscopic analysis, it is desirable to minimize the amount of ammonium salt in the fractions that are obtained from liquid chromatography. High concentrations of ammonium salt can impair the ionization efficiency of the mass spectrometer, particularly when the mass spectrometer operates by electrospray ionization. Ammonium salts typically display water solubility so long as the R groups that form the ammonium salts are not, as a whole, too hydrophobic. In various aspect the ammonium salt is selected so as to provide an aqueous solution with a pH in the range of from 5.0 to 9.0, or 5.5 to 8.5, or 6.0 to 8.0, or 6.5 to 7.5.

In various aspects an R group is, independently at each occurrence within an ammonium molecule, and for each of primary, secondary and tertiary ammonium salts, hydrocarbon, i.e., the R group is formed entirely of hydrogen and carbon; hydrocarbons with 1-10 carbons; hydrocarbons with 1-6 carbons; hydrocarbons with 5-8 carbons; selected from alkyl and cycloalkyl groups; selected from methyl, ethyl, propyl (including geometric isomers thereof), butyl (including geometric isomers thereof), pentyl (including geometric isomers thereof), hexyl (including geometric isomers thereof), cyclohexyl, and cycopentyl (including methyl-substituted cyclopentyl); selected from organic groups having an atomic mass of 15-250, which may or may not include atoms other than carbon and hydrogen; halide substituted in one occurrence; and/or halide substituted in two occurrences; where an R group may according to the present invention embody any two or more of these features in every combination so long as two features are not contradictory. As referred to herein, alkyl and cylcoalkyl groups may contain unsaturation, e.g., a double bond, however cycloalkyl does not include aromatic rings. Nevertheless, in one aspect the R group is an aromatic ring selected from phenyl and C₁-C₆alkyl substituted phenyl. In another aspect, the R group is an alkyl group having aryl substitution, e.g., benzyl. In one aspect, the ammonium salt is a secondary or tertiary ammonium salt having alkyl or cycloalkyl groups with 5-8 carbons. In one aspect, the ammonium salt contains a cationic, i.e., protonated form of triethylamine, diallylamine, diisopropylamine, N,N-dimethyl-N-cyclohexylamine, or N,N-dimethyl-N-isopropylamine. In one aspect, the ammonium salt is the salt form of N-N-dimethylaminobutane. In another aspect, the ammonium salt is the salt form of N,N-dimethylcyclohexylamine. In another aspect the ammonium salt is the salt form of triethylamine.

The selection of the amino component of the ammonium salt can have an effect on the signal generated by the nucleic acid molecule using liquid chromatography followed by mass spectroscopy. For instance, while dimethylaminobutane and dimethylcyclohexylamine provide similar responses, triethylamine decreases response as observed by mass spectrometry following liquid chromatography, by about 25% compared to dimethylaminobutane.

In one aspect, the protic acid of the ammonium salt is of the formula Ra-COOH so that the anionic counterion to the ammonium group has the formula Ra-COO⁻. In various aspects, Ra is hydrogen (i.e., the counterion is derived from formic acid) methyl, ethyl, propyl, selected from methyl and ethyl, or selected from methyl, ethyl and propyl, and mono- and poly-halogenated versions thereof. In another aspect, the protic acid used to form the ammonium salt is a protonated form of the carbonate family of anions, i.e., bicarbonate (HCO₃) and/or carbonate (CO₃) is the anionic counterion to the ammonium group. In other aspects of the invention, the protic acid is an inorganic acid, e.g., HCl and HBr. Generally, in order of preference, the protic acid of the ammonium salt is acetic acid, formic acid, carbonic acid (so as to provide bicarbonate anion), or hydrogen chloride.

In one aspect of the invention, the ammonium group is a tertiary ammonium group having R groups as defined above, and the counterion is acetate (not including halogenated acetate), carbonate/bicarbonate, or is selected from acetate (not including halogenated acetate) and carbonate/bicarbonate. When the counterion is acetate, exemplary ammonium salts include, without limitation, triethylamine acetate, dimethylbutamine acetate, dimethylisopropylamine acetate, dimethylhexylamine acetate, dimethylcyclohexylamine acetate, and disopropylamine acetate.

Beside the selection of Buffers A and B, the conditions under which the liquid chromatography is operated are important in being able to resolve, or distinguish, two nucleic acid structures of identical length but of somewhat different nucleotide base sequence. For example, generally the liquid chromatographic analysis of the present invention is carried out at either room temperature, i.e., about 25° C., or at elevated temperature. Typically, elevated temperatures are less than 75° C. In various aspects, the chromatography column is maintained within the following temperature ranges during the chromatography: 20° C.-80° C., 25° C.-70° C., 25° C.-65° C., 25° C.-60° C., 30° C.-80° C., 30° C.-70° C., 30° C.-65° C., 30° C.-60° C., 30° C.-55° C., 30° C.-50° C., or 30° C.-45° C. Elevated temperature is typically desirable because it may provide a chromatogram wherein the peaks are higher, narrower, and display greater resolution. However, as the temperature exceeds about 70° C., bubble formation in the eluent is sometimes observed and this may cause a loss in resolution.

As mentioned above, the pH of the elution buffer is also an important factor in the successful chromatographic analysis. Generally, the elution buffer maintains a pH ranging from about 5.0 to 9.0, as discussed above. Typically, retention time is unaffected so long as the pH of the elution buffer is maintained within this pH range.

The chromatography is typically run under pressure, ie., pressure is used to push the elution buffer through the column. As the pressure is increased there is typically an increase in the flow rate of the buffer through the column. A pressure ranging from about 200-1600 bars, and a flow rate ranging from about 10 μL to 2000 μL per minute are typically suitable conditions for operating the column chromatography.

The length and stationary phase of the liquid chromatography column are two other parameters that must be selected. Typically, the column is 18-500 mm in length. In various aspects, the column is 18, 25, 50, 100, 250, or 500 mm in length and can be at a micro-, or macro-scale. The stationary phase is selected so as to provide a reverse phase column, i.e., a column having a hydrophobic phase surrounding the solid phase. A suitable reverse phase column is the MICROSORB™ C18 column from Varian Inc. (Palo Alto, Calif.; www.varianinc.com). This column is a monomeric silica column, with 5 micron spherical particles, 300 Å pores, C18 stationary phase, 12% carbon load and is endcapped. A substantially equivalent column is the JUPITER™ C18 column made by Phenomenex U.S.A. (Torrance, Calif.; www.phenomenex.com). A similar column is the XTERRA™ column from Waters (Milford, Mass.: www.waters.com) which contains a hybrid particle made of silica and polymer to extend pH stability with a pore size of 120 Å and particle size from 2.5 micron to 5 micron. Columns having a completely polymeric solid support may also be used. In one preferred aspect of the invention, the reverse phase column has a C18 stationary phase and a pore size of at least 120 Å. Endcapping of silica columns is desirable in order to minimize tailing and improve peak shape, and accordingly a preferred column has this feature. The carbon load of the column is important to ensure sufficient retention (carbon load does not apply to polymeric columns), and is preferably in the range of 5-20%. The particle size typically varies from about 2 microns up to about 10 microns, where these are typical sizes for columns that are currently commercially available. Smaller particle size generally result in improved chromatography, so particle sizes smaller than 5 μM are preferred. Column dimensions are not critical, but may be chosen based on scale and type of analysis. Generally for fast, higher throughput analysis of small samples (<25 μL or 250 μg of analyte) a small column is preferred, such as the 2.1×15 mm (diameter×length) XTERRA™ column with 2.5 μM particles allowing a complete run in four minutes at a flow rate of 250 μL/min. When injecting larger samples sizes it is typically preferred to use a larger column, where larger columns are about 4.6 mm in diameter and about 250 mm in length. Columns having dimensions from 0.3 mm to 4.6 mm in internal diameter and from 10 nun to 250 mm long are available from many commercial suppliers (e.g., Water) and are suitably used in the present invention. A typical LCMS column will be 1 mm×50 mm. The flow rate will be dependent on the column dimensions and will vary from a few microliters per minute for a 0.3 mm ID column up to about 1500-2000 microliters per minute for a 4.6 mm ID column.

As explained above, the mobile phase is preferably formed by incrementally combining two different solutions (Buffers A and B). The salt is preferably present in the solution at a concentration of 1 mM to 200 mM, more preferably at a concentration of 1 mM to 100 mM, still more preferably at a concentration of 5 mM to 50 mM. Particularly when the liquid chromatographic analysis is followed by mass spectroscopic analysis, lower salt concentration is preferred, and in such a situation a salt concentration of about 5 mM is preferred. Buffer B preferably contains 10-90% (volume/volume) polar organic molecules (e.g., acetonitrile, methanol, or isopropanol) in Buffer A. Buffers A and/or B may contain optional components, and in one aspect an optional component present in Buffer A is also present in Buffer B. A suitable optional component is EDTA, where EDTA may be used at a concentration of about 0.1 mM in the buffers.

For instance, liquid chromatography according to the present invention may be performed as follows. First, a shallow gradient of acetonitrile or other suitable solvent may be used to elute the nucleic acid molecules and provide for sample clean up. For instance, when using acetonitrile, the gradient can start at, or about at, 5% and increase to, or about to, 20%, where these percent values refer to the volume percent of organic solvent in water as the elution buffer. Methanol can be used in place of acetonitrile, but the use of methanol may require increasing the final gradient composition to 50% or even 75% methanol. The fraction of solvent required depends partly on the column used, and also on the length range of nucleic acid molecules being analyzed. Generally a strong solvent wash is applied to the column at the end of the run to elute any large, hydrophobic components. In a preferred embodiment, the analysis portion of the gradient starts at 5% acetonitrile and increases to 15% over about 90 seconds, where this is followed by a wash which quickly pushes a “plug” of 45% acetonitrile onto the column for just a few seconds followed by a return to starting conditions of 5% acetonitrile. A preferred buffer system incorporates 5 mM N,N-dimethylaminobutyl acetate, and operates at pH 7. Concentrations of ammonium salts from 1 mM up to 50 mM are observed to have equivalent response in a mass spectrometer (e.g., there is no evidence of ion suppression within this range) but variation of ammonium salt concentration within this range may have slight effects on the retention times of the nucleic acid molecules. Variation in retention times may be compensated for by adjusting the solvent composition during the LC run. As mentioned above, the pH range is flexible, where a range from pH 6 to pH 8 can be used with little or no noticeable change in outcome, i.e., little or no effect on nucleic acid retention times.

In order to conveniently prepare and/or use Buffers A and B, and to perform chromatographic separation of nucleic acid molecules, and to prepare suitable short nucleic acid molecules to be separated by column chromatography according to the present invention, in further aspects the present invention provides kits. In one aspect, the kit includes a container holding ammonium salt and a separate container holding organic solvent. The container of ammonium salt and/or the container of organic solvent may include optional ingredients, e.g., EDTA and/or water. In one aspect, the container of ammonium salt and the separate container of organic solvent each contain water so that the ammonium salt is present in a dissolved form in the kit. In another aspect, the water is held in a separate container. In another aspect the kits includes a container that holds all the ingredients necessary to make Buffer A, except that water is not present in the container, and also separately includes a container that holds all the ingredients necessary to make Buffer B, except that water is not present in the container. Water may, or may not, be included in a separate container that is found within the kit. The water is preferably HPLC grade water.

Preferred kits of the present invention contain one, each combination of two, each combination of three, each combination of four, or all five of the following components:

1. A container holding components comprising ammonium salt dissolved in water, where the water should preferably be HPLC grade water. The ammonium salt may be any of the one or more ammonium salts identified above. For example, in one aspect, the ammonium salt is N,N-dimethyl-N-butylammonium with a counterion, where, in a further aspect, the counterion is acetate. In a preferred embodiment the ammonium salt is present in the water at a concentration of about 1-200 mM, preferably about 1-100 mM, preferably about 5-50 mM, and more preferably about 5 mM. The container may hold ammonium salt at a concentration greater than 100 mM, but in such case the components in this container will probably need to be diluted with water in order to form an effective buffer. This solution of ammonium salt and water preferably has a pH of about 7.0-7.5, and is typically about 7.2, and is referred to herein as Buffer A. In one aspect, this container holds only ammonium salt dissolved in water.

2. A container holding components comprising ammonium salt, water and organic solvent. In one aspect, the components comprise Buffer A and organic solvent, where the mixture is preferably homogeneous, i.e., the organic solvent dissolves or is miscible in Buffer A. In one aspect the organic solvent is acetonitrile. In another aspect the organic solvent is methanol. In various aspects, the organic solvent constitutes, on a volume percent basis, based on the total volume of the components in the container, 15%-20%, 20%-25%, 25%-30%, 30%-35%, 35%-40%, 40%-45%, 45%-50% or 50%-55% or 55-60% or 65-70% or 75-80%. In a preferred embodiment the organic solvent constitutes 25% acetonitrile. In another preferred embodiment the organic solvent constitutes 50% acetonitrile. Thus, a preferred component is a solution of 75 vol % Buffer A and 25 vol % acetonitrile, while another preferred component is a solution of 50 vol % Buffer A and 50 vol % acetonitrile.

3. A chromatography column, preferably a reverse phase chromatography column as described above, may be included in the kit. Thus, a preferred reverse phase chromatography is a C18 reverse phase chromatography column. A preferred C18 reverse phase column has a pore size of at least 120 Å. A preferred C18 reverse phase column has a particle size of 2 microns to 10 microns. A preferred column size is 0.3 mm to 4.6 mm in inner diameter and from 10 mm to 250 mm in length.

4. An instruction booklet. The instructions booklet will provide useful information for running the column chromatographic analysis. Useful information may include a description of how to program a chromatogram using the two Buffers of the present invention.

5. A container holding water. The water is preferably HPLC grade water.

The kit may optionally include one or more components useful in preparing the short nucleic acid molecules that will be characterized by the chromatographic technique. For instance, the kit may include primers as described herein, restriction enzymes as described herein, and/or polymerase and other components necessary for amplification of nucleic acid.

Preferred exemplary conditions for liquid chromatographic analysis are described in detail in Examples below. In various aspects of the invention, two nucleic acid molecules are separated by liquid chromatography, where these two molecules each have 4, or 5, or 6, or 7, or 8, or 9, or 10, or 11, or 12, or 13, or 14, or 15, or 16, or 17, or 18 nucleotides and, furthermore, these two nucleic acid molecules either have the same base sequence of nucleotides with one exception, or they are made from the same nucleotides but these nucleotides are arranged in a different sequence. Typically, as the number of nucleotides present in a nucleic acid molecule increases, it becomes increasingly difficult to get the nucleic acid molecule to elute from the column. For this reason, higher temperature and/or higher organic solvent concentration are generally preferred when the nucleic acid molecule contains much more than about 8-10 nucleotides.

For instance, the two nucleic acid molecules may have the sequences 5′-A-G-C-T-A-3′ and 5′-A-G-A-T-A-3′, where this is an example of two nucleic acid molecules each having 5 nucleotides where the two nucleic acid molecule have the same base sequence of nucleotides with one exception (C vs. A in the third position from the 5′ end). As another example, the two nucleic acid molecules may have the sequences 5′-A-C-G-T-A-3′ and 5′-A-G-C-T-A-3′, where this is an example of two nucleic acid molecules each having 5 nucleotides where the two nucleic acid molecules are made from the same nucleotides but these nucleotides are arranged in a different sequence.

In one aspect of the invention, the two nucleic acid molecules are composed of identical nucleotides, but the order of the nucleotides in the two nucleic acid molecules is non-identical. In another aspect, the two nucleic acid molecules each have the sequence 5′-n-X-m⁻³′, where n and m each represent a sequence of from 0-10 nucleotides, and X represents a single nucleotide, and the two nucleic acid molecules each have the same sequences n and m, but differ in the identify of the nucleotide at location X.

Thus, the present invention provides a method of performing liquid chromatography comprising applying two nucleic acid molecules to a liquid chromatography column, where the two nucleic acid molecules have an identical number of nucleotide bases, but have different nucleotide sequences; and eluting the two nucleic acid molecules from the column with an elution buffer so that the two nucleic acid molecules have different elution times; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid. In this method, while it is necessary to apply at least two unique nucleic acid molecules to the liquid chromatography column, it should be mentioned that more than two unique nucleic acid molecules may be applied to a liquid chromatography column. For instance, four nucleic acid molecules may be applied to the column, where those four nucleic acid molecules consist of two pairs of nucleic acid molecules, each pair consisting of two nucleic acid molecules that have the same number of nucleotide bases, but have different nucleotide sequences.

The above description generally discloses a method for identifying a nucleotide at a defined position in a target nucleic acid, which is particularly useful in genotyping a single nucleotide polymorphism (SNP). However, in another embodiment, the present invention may also be used in genotyping genetic variations in a defined location of a wild type sequence resulting from insertions, deletions or substitutions involving more than one nucleotide. Generally, the insertions, deletions or substitutions involve up to 20 nucleotides, and typically involve 1, 2, 3, 4 or 5 nucleotides.

The term “single nucleotide polymorphism,” as used herein, refers to any nucleotide sequence variation that involves a single nucleotide, preferably one that is common in a population of organisms and is inherited in a Medelian fashion. Typically, an SNP is either of two possible nucleotides, and there is no possibility of finding a third or fourth nucleotide identity at an SNP site. A “wild type” sequence, as used herein, refers to a nucleotide sequence that is most popular in a selected population of an organism from which the sequence may be isolated.

In certain embodiments wherein a genetic variation (an SNP or a genetic variation involving more than one nucleotide) at a defined location is to be genotyped, the genetic variation may be characterized first. In other words, the sequence containing the genetic variation may be determined first. To determine whether a particular individual contains a wild-type sequence or a sequence with the genetic variation, (1) a nucleic acid fragment from the individual, (2) a wild type sequence, and (3) a sequence having the pre-determined genetic variation are then individually used as a template for amplification using specifically designed ODNPs as described above. The resulting products from the three separate amplification reactions are then separately digested with restriction endonuclease(s) that recognize the recognition site(s) created from the recognition sequences in the ODNPs. The short digestion product from each digestion reaction is subsequently subjected to liquid chromatographic analysis. The result of this analysis using the nucleic acid fragment from the individual as the amplification template (“Result 1”) is compared with that using a wild type sequence (“Result 2”) or a sequence containing the genetic variation (“Result 3”) as the template. If Result 1 is the same as Result 2, the individual has a wild type genotype with respect to that defined position. If Result 1 is the same as Result 3, the individual has the predetermined genetic variation at the defined position.

The present method may also be used to determine whether a subject organism (e.g., a person) is homozygous or heterozygous as to a particular genomic fragment or gene. If a person is homozygous with respect to that genomic fragment or gene, there will be two peaks corresponding to the two single-stranded DNA fragments resulting from the denaturation of a short double-stranded digestion fragment detected by liquid chromatography. If the person is heterozygous with respect to that genomic fragment or gene, there will be four peaks detected corresponding to four single-stranded DNA fragments resulting from the denaturation of two short double-stranded digestion fragments: one corresponding to one allele from one parent, the other from the other parent.

Although the above description refers to target nucleic acids as double-stranded nucleic acids, one of ordinary skill in the art would readily appreciate that the present invention is applicable to single-stranded target nucleic acids as well. For instance, a single-stranded target nucleic acid may be used as a template for synthesizing its complementary strand and thus becomes double-stranded nucleic acid before forming a mixture with an ODNP pair of the present invention. Alternatively, a single-stranded target nucleic acid may be directly mixed with the ODNPs of the present invention and its complementary strand is produced using one of the ODNPs as a primer during the amplification reaction for producing a nucleic acid containing the nucleotide(s) at a defined location.

Thus, the present invention provides, in one aspect, a method for identifying one or more nucleotide bases at a defined position of a polynucleotide, where the position is defined relative to known nucleotide base sequences at both a 3′ and 5′ direction from the defined position, comprising:
(1) combining the polynucleotide with a first oligonucleotide primer (ODNP), the first ODNP comprising
   (a) a nucleotide base sequence that is a recognition sequence for a restriction endonuclease, the restriction endonuclease having a cleavage site outside the recognition sequence,
   (b) a nucleotide base sequence that enables the first ODNP to anneal to the polynucleotide at a location 3′ to the defined position, and
   (c) a 3′ end that may be extended by a polymerase under primer extension conditions;
(2) extending the first ODNP to incorporate the complement of the base(s) at the defined position, and additional bases 5′ to the defined position, so as to provide an extended first ODNP;
(3) combining a second ODNP with either or both of the extended first ODNP or the complement of the polynucleotide (where the complement would typically be present because the polynucleotide was originally double-stranded), the second ODNP comprising
   (a) a nucleotide base sequence that is a recognition sequence for a restriction endonuclease, the restriction endonuclease having a cleavage site outside the recognition sequence,
   (b) a nucleotide base sequence that enables the second ODNP to anneal to the complement of the polynucleotide at a position 3′ to the complement of the defined position, and
   (c) a 3′ end that may be extended by a polymerase under primer extension conditions;
(4) extending the second ODNP to incorporate the bases of the defined position and additional bases 5′ to the defined position, so as to provide an extended second ODNP
(5) combining the extended first and the extended second ODNPs, with or without the amplification product thereof as formed from additional first and second ODNPs acting on the extended first and the extended second ODNPs as template nucleic acids for primer extension, with restriction enzymes that recognize the recognition sequences, under conditions where the restriction enzyme(s) cleave a double-stranded fragment comprising the defined location and the complement thereof, so as to provide the bases to be identified in a short nucleic acid molecule (either a short oligonucleotide or a short polynucleotide);
(6) characterizing the short nucleic acid molecule by liquid chromatography, so as to identify the one or more nucleotide bases at the defined position.

The above method may also be applied to oligonucleotides, preferably oligonucleotides having more than 50 bases.

In another aspect, the present invention provides a method for identifying one or more nucleotide bases at a defined position of a polynucleotide, where the polynucleotide is preferably DNA, and more preferably a cDNA or genomic DNA, and where the position is defined relative to known nucleotide base sequences at both a 3′ and 5′ direction from the defined position, comprising:
amplifying the polynucleotide from a subject using a pair of primers (as described above, or as describe below) to thereby form amplified polynucleotide;
digesting the amplified polynucleotide with a restriction endonuclease to form a short nucleic acid molecule; and
characterizing the short nucleic acid molecule by liquid chromatography, so as to identify the one or more nucleotide bases at the defined position.

The pair of primers were described above. In a further aspect, the pair of primers may be described as follows: each primer comprises a linear oligonucleotide comprising a 5′ and a 3′ end, said oligonucleotide consisting of at least 35 nucleotides, wherein a first portion of said oligonucleotide of at least 13 nucleotides at the 5′ end of said oligonucleotide and a second portion of the oligonucleotide of from 5 to 22 nucleotides at the 3′ end of the oligonucleotide are at least substantially complementary to a first portion and a second portion of the polynucleotide, wherein 4-8 nucleotides between the first and second portions of each oligonucleotide comprise a recognition sequence for a restriction enzyme that cleaves at least 5 nucleotides from its recognition site, wherein the segment of the polynucleotide (e.g., cDNA or genomic DNA) does not comprise the recognition site for the restriction endonuclease, wherein each primer of the pair of primers is complementary to an opposite strand of a double stranded polynucleotide, wherein the pair of primers is complementary to two non-contiguous portions of the double stranded polynucleotide, wherein 1 to 20 nucleotides separate the two non-contiguous portions of the double stranded polynucleotide. See also the primers disclosed in PCT publication No. WO 00/31300, the entire disclosure of which is incorporated herein by reference for all purposes.

To summarize specific aspects of the invention, the present invention provides the following:

A method of performing liquid chromatography comprising: applying two nucleic acid molecules to a liquid chromatography column, where the two nucleic acid molecules have an identical number of nucleotide bases, but have different nucleotide sequences; and eluting the two nucleic acid molecules from the column with an elution buffer so that the two nucleic acid molecules have different elution times; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid. Each of the following is an optional embodiment of the invention, where the invention also provides combinations of the following optional embodiments that are not inconsistent with each other: the two nucleic acid molecules are composed of identical nucleotides, but the order of the nucleotides in the two nucleic acid molecules is non-identical; the two nucleic acid molecules each have the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X; the nucleic acid molecules have 3 nucleotides; the nucleic acid molecules have 4 nucleotides; the nucleic acid molecules have 5 nucleotides; the nucleic acid molecules have 6 nucleotides; the nucleic acid molecules have 7 nucleotides; the nucleic acid molecules have 8 nucleotides; the nucleic acid molecules have 9 nucleotides; the nucleic acid molecules have 10 nucleotides; the nucleic acid molecules have 11 nucleotides; the nucleic acid molecules have 12 nucleotides; the nucleic acid molecules have 13 nucleotides; the nucleic acid molecules have 14 nucleotides; the nucleic acid molecules have 15 nucleotides; the nucleic acid molecules have 16 nucleotides; the nucleic acid molecules have 17 nucleotides; the liquid chromatography column is a reverse phase chromatography column; the liquid chromatography column is a C18 reverse phase chromatography column; the liquid chromatography column is a C18 reverse phase chromatography column having a pore size of at least 120 Å; the liquid chromatography column is a C18 reverse phase chromatography column having a pore size of at least 120 Å and a particle size of the solid support within the column is 2 microns to 10 microns; the liquid chromatography column is a C18 reverse phase chromatography column containing monomeric silica; the liquid chromatography column is a C18 reverse phase chromatography column having 5-20% carbon load; the liquid chromatography column is maintained at a temperature between 20° C.-80° C. during at least part of the time the nucleic acid molecules are eluting through the column; the liquid chromatography column is maintained at a temperature between 30° C.-70° C. during at least part of the time the nucleic acid molecules are eluting through the column; Buffer A comprises water and an ammonium salt of a secondary or tertiary amine complexed with an organic or inorganic acid; Buffer A has a pH ranging from 5-9; Buffer A has a pH ranging from 6-8; the ammonium salt is present in Buffer A at a concentration of 1-100 mM; the ammonium salt is present in Buffer A at a concentration of 5-50 mM; the ammonium salt includes the protonated form of a secondary amine of the formula R₂NH and R at each occurrence is independently selected from C₁-C₁₀hydrocarbon groups, and the hydrocarbon is optionally an alkyl or cycloalkyl group; the ammonium salt is includes the protonated form of a secondary amine of the formula R₂NH and R at each occurrence is independently selected from C₁-C₆hydrocarbon groups, where the hydrocarbon of the amine group is optionally selected from alkyl and cycloalkyl groups; the ammonium salt is the protonated form of a secondary amine of the formula R₂NH, and R at each occurrence is independently selected from C₅-C₈hydrocarbon groups, and the hydrocarbon is optionally selected from alkyl and cycloalkyl groups; the amine component of the ammonium salt is a secondary amine selected from diallylamine and diisopropylamine; the ammonium salt is the protonated form of a tertiary amine of the formula R₃N and R at each occurrence is independently selected from C₁-C₁₀hydrocarbon groups, where optionally the hydrocarbon is selected from alkyl and cycloalkyl groups; the ammonium salt is the protonated form of a tertiary amine of the formula R₃N and R at each occurrence is independently selected from C₁-C₁₀hydrocarbon groups, and optionally the hydrocarbon is selected from alkyl and cycloalkyl groups; the ammonium salt is the protonated form of a tertiary amine of the formula R₃N and R at each occurrence is independently selected from C₁-C₁₀hydrocarbon groups, and optionally the hydrocarbon is selected from alkyl and cycloalkyl groups; the amine component of the ammonium salt is selected from triethylamine, N,N-dimethyl-N-cyclohexylamine, N,N-dimethyl-N-isopropylamine, and N,N-dimethyl-N-butylamine; the secondary or tertiary amine is complexed with, i.e., is the reaction product of, an organic acid; the secondary or tertiary amine is complexed with, i.e., is the reaction product of, an organic acid, and the organic acid is selected from acetic acid, propionic acid, and halogenated versions thereof; the secondary or tertiary amine is complexed with an organic acid, and the organic acid is acetic acid; the secondary or tertiary amine is complexed with an organic acid, and the organic acid is formic acid; the secondary or tertiary amine is complexed with an organic acid, and the organic acid is an acid form of carbonate; the secondary or tertiary amine is complexed with an organic acid, and the organic acid is an acid form of bicarbonate; the secondary or tertiary amine is complexed with an inorganic acid, and the inorganic acid is hydrochloric acid; Buffer B comprises organic solvent and Buffer A; Buffer B has a pH ranging from 5-9; Buffer A has a pH ranging from 6-8; the ammonium salt is present in Buffer A at a concentration of 1-100 mM; the ammonium salt is present in Buffer A at a concentration of 5-50 nM; the organic solvent comprises acetonitrile; the organic solvent comprises methanol; and Buffer B comprises organic solvent and Buffer A in a organic solvent:Buffer A volume ratio of 25-75:75-25.

A method of performing liquid chromatography comprising: applying two nucleic acid molecules to a liquid chromatography column, where the two nucleic acid molecules have an identical number of nucleotide bases within the range of 2-10, but have different nucleotide sequences, and the liquid chromatography column is a reverse phase chromatography column; and eluting the two nucleic acid molecules from the column with an elution buffer so that the two nucleic acid molecules have different elution times; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with, i.e., is the reaction product of, an organic or inorganic acid. Each of the following is an optional embodiment of the invention, where the invention also provides combinations of the following optional embodiments that are not inconsistent with each other: the two nucleic acid molecules are composed of identical nucleotides, but the order of the nucleotides in the two nucleic acid molecules is non-identical; the two nucleic acid molecules each have the sequence 5′-n-X-m-3′, where n and m each represent a sequence of from 0-9 nucleotides, and X represents a single nucleotide, and the two nucleic acid molecules each have the same sequences n and m, but differ in the identify of the nucleotide at location X; the nucleic acid molecules have 3 nucleotides; the nucleic acid molecules have 4 nucleotides; the nucleic acid molecules have 5 nucleotides; the nucleic acid molecules have 6 nucleotides; the nucleic acid molecules have 7 nucleotides; the nucleic acid molecules have 8 nucleotides; the nucleic acid molecules have 9 nucleotides; the nucleic acid molecules have 10 nucleotides; the nucleic acid molecules have 11 nucleotides; the nucleic acid molecules have 12 nucleotides; the nucleic acid molecules have 13 nucleotides; the nucleic acid molecules have 14 nucleotides; the nucleic acid molecules have 15 nucleotides; the nucleic acid molecules have 16 nucleotides; the nucleic acid molecules have 17 nucleotides; the liquid chromatography column is a C18 reverse phase chromatography column; the liquid chromatography column is a C18 reverse phase chromatography column having a pore size of at least 120 Å; the liquid chromatography column is a C18 reverse phase chromatography column having a pore size of at least 120 Å and a particle size of the solid support within the column is 2 microns to 10 microns; the liquid chromatography column is a C18 reverse phase chromatography column containing monomeric silica; the liquid chromatography column is a C18 reverse phase chromatography column having 10-15% carbon load; the liquid chromatography column is maintained at a temperature between 20° C.-80° C. during at least part of the time the nucleic acid molecules are eluting through the column; the liquid chromatography column is maintained at a temperature between 30° C.-70° C. during at least part of the time the nucleic acid molecules are eluting through the column; Buffer A comprises water and an ammonium salt of a secondary or tertiary amine reacted with, i.e., complexed with, an organic or inorganic acid; Buffer A has a pH ranging from 5-9; Buffer A has a pH ranging from 6-8; the ammonium salt is present in Buffer A at a concentration of 1-100 mM; the ammonium salt is present in Buffer A at a concentration of 5-50 mM; the ammonium salt includes the protonated form of a secondary amine of the formula R₂NH and R at each occurrence is independently selected from C₁-C₁₀hydrocarbon groups, and the hydrocarbon is optionally an alkyl or cycloalkyl group; the ammonium salt is includes the protonated form of a secondary amine of the formula R₂NH and R at each occurrence is independently selected from C₁-C₆hydrocarbon groups, where the hydrocarbon of the amine group is optionally selected from alkyl and cycloalkyl groups; the ammonium salt is the protonated form of a secondary amine of the formula R₂NH, and R at each occurrence is independently selected from C₅-C₈hydrocarbon groups, and the hydrocarbon is optionally selected from alkyl and cycloalkyl groups; the amine component of the ammonium salt is a secondary amine selected from diallylamine and diisopropylamine; the ammonium salt is the protonated form of a tertiary amine of the formula R₃N and R at each occurrence is independently selected from C₁-C₁₀hydrocarbon groups, where optionally the hydrocarbon is selected from alkyl and cycloalkyl groups; the ammonium salt is the protonated form of a tertiary amine of the formula R₃N and R at each occurrence is independently selected from C₁-C₁₀hydrocarbon groups, and optionally the hydrocarbon is selected from alkyl and cycloalkyl groups; the ammonium salt is the protonated form of a tertiary amine of the formula R₃N and R at each occurrence is independently selected from C₁-C₁₀hydrocarbon groups, and optionally the hydrocarbon is selected from alkyl and cycloalkyl groups; the amine component of the ammonium salt is a secondary amine selected from diallylamine and diisopropylamine; the ammonium salt is the protonatead form of a tertiary amine of the formula R₃N and R at each occurrence is independently selected from C₁-C₁₀hydrocarbon groups, where optionally the hydrocarbon is selected from alkyl and cycloalkyl groups; the ammonium salt is the protonated form of a tertiary amine of the formula R₃N and R at each occurrence is independently selected from C₁-C₁₀hydrocarbon groups, where optionally the hydrocarbon is selected from alkyl and cycloalkyl groups; the ammonium salt is the protonated form of a tertiary amine of the formula R₃N and R at each occurrence is independently selected from C₁-C₁₀hydrocarbon groups, where optionally the hydrocarbon is selected from alkyl and cycloalkyl groups; the amine component of the ammonium salt is selected from triethylamine, N,N-dimethyl-N-cyclohexylamine, N,N-dimethyl-N-isopropylamine, and N,N-dimethyl-N-butylamine; the secondary or tertiary amine is complexed with, i.e., is the reaction product of, an organic acid; the secondary or tertiary amine is complexed with an organic acid, and the organic acid is selected from acetic acid, propionic acid, and halogenated versions thereof; the secondary or tertiary amine is complexed with an organic acid, and the organic acid is acetic acid; the secondary or tertiary amine is complexed with an organic acid, and the organic acid is formic acid; the secondary or tertiary amine is complexed with an organic acid, and the organic acid is an acid form of carbonate; the secondary or tertiary amine is complexed with an organic acid, and the organic acid is an acid form of bicarbonate; the secondary or tertiary amine is complexed with an inorganic acid, and the inorganic acid is hydrochloric acid; Buffer B comprises organic solvent and Buffer A; Buffer B has a pH ranging from 5-9; Buffer A has a pH ranging from 6-8; the ammonium salt is present in Buffer A at a concentration of 1-100 mM; the ammonium salt is present in Buffer A at a concentration of 5-50 mM; the organic solvent in Buffer B comprises acetonitrile; the organic solvent in Buffer B comprises methanol; and Buffer B comprises organic solvent and Buffer A in a organic solvent:Buffer A volume ratio of 25-75:75-25.

A method of performing liquid chromatography comprising: applying two nucleic acid molecules to a liquid chromatography column, where the two nucleic acid molecules have an identical number of nucleotide bases, but have different nucleotide sequences; and eluting the two nucleic acid molecules from the column with an elution buffer so that the two nucleic acid molecules have different elution times; the elution buffer being formed from Buffer A and Buffer B, where Buffer A comprises water and an ammonium salt that is formed from a secondary or tertiary amine complexed with an organic or inorganic acid; and Buffer B comprises water, organic solvent, and an ammonium salt that is formed from a secondary or tertiary amine complexed with an organic or inorganic acid; where elution buffer of incrementally increasing organic solvent concentration is applied to the column. Each of the following is an optional embodiment of the invention, where the invention also provides combinations of the following optional embodiments that are not inconsistent with each other: the two nucleic acid molecules are composed of identical nucleotides, but the order of the nucleotides in the two nucleic acid molecules is non-identical; the two nucleic acid molecules each have the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X; and the two nucleic acid molecules each have 2-10 nucleotides.

A method of performing liquid chromatography and mass spectrometric analysis comprising: applying a plurality of pairs of nucleic acid molecules to a liquid chromatography column, where each pair of nucleic acid molecules is formed from two nucleic acid molecules that have an identical number of nucleotide bases, but have different nucleotide sequences; eluting the plurality of pairs of nucleic acid molecules from the column with an elution buffer so that the two nucleic acid molecules that form each pair have different elution times; and characterizing each nucleic acid molecule by mass spectroscopy; the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid. Each of the following is an optional embodiment of the invention, where the invention also provides combinations of the following optional embodiments that are not inconsistent with each other: each member of a pair of two nucleic acid molecules is composed of identical nucleotides, but the order of the nucleotides in the two nucleic acid molecules is non-identical; each member of a pair of two nucleic acid molecules has the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X; the liquid chromatography column is a reverse phase chromatography column; the liquid chromatography column is a C18 reverse phase chromatography column; the liquid chromatography column is a C18 reverse phase chromatography column having a pore size of at least 120 Å; the liquid chromatography column is a C18 reverse phase chromatography column having a pore size of at least 120 Å and a particle size of the solid support within the column is 2 microns to 10 microns; the liquid chromatography column is a C18 reverse phase chromatography column containing monomeric silica; the liquid chromatography column is a C18 reverse phase chromatography column having 5-20%, or 10-15% carbon load; the liquid chromatography column is maintained at a temperature between 20° C.-80° C. during at least part of the time the nucleic acid molecules are eluting through the column; the liquid chromatography column is maintained at a temperature between 30° C.-70° C. during at least part of the time the nucleic acid molecules are eluting through the column.

A composition referred to as Buffer B comprising water, the reaction product of secondary or tertiary amine with organic or inorganic acid, and organic solvent. Optionally, the reaction product of secondary or tertiary amine with organic or inorganic acid is selected from an acetate salt of an amine selected from the group consisting of triethylamine, diallylamine, diisopropylamine, N,N-dimethyl-N-cyclohexylamine, N,N-dimethyl-N-isopropylamine, and N,N-dimethyl-N-butylamine. Optionally this composition has a pH of 6-8. Optionally the organic solvent is selected from methanol and acetonitrile. Optionally, the composition has a water:organic solvent ratio of 95-25:5-75. Optionally the reaction product is present in Buffer B at a concentration of 1-100 mM.

A composition comprising water, the reaction product of secondary or tertiary anine with organic or inorganic acid, organic solvent, and two nucleic acid molecules, where the two nucleic acid molecules have an identical number of nucleotide bases, but have different nucleotide sequences. Optionally, the two nucleic acid molecules are composed of identical nucleotides, but the order of the nucleotides in the two nucleic acid molecules is non-identical; optionally, the two nucleic acid molecules each have the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X; optionally the two nucleic acid molecules have 4-10 nucleotides; optionally the reaction product of secondary or tertiary amine with organic or inorganic acid is selected from an acetate salt of an amine selected from the group consisting of triethylamine, diallylamine, diisopropylamine, N,N-dimethyl-N-cyclohexylamine, N,N-dimethyl-N-isopropylamine, and N,N-dimethyl-N-butylamine; optionally the composition has a pH of 6-8; optionally the organic solvent is selected from methanol and acetonitrile; optionally the composition has a water:organic solvent ratio of 95-25:5-75. Optionally, the composition comprises four nucleic acid molecules, the four nucleic acid molecules being two pairs of nucleic acid molecules, each pair of nucleic acid molecules being formed from two nucleic acid molecules that have an identical number of nucleotide bases, but have different nucleotide sequences.

A kit for chromatographic analysis comprising: (a) a container holding components comprising water and the reaction product of, i.e., the salt of, secondary or tertiary amine with organic or inorganic acid (Buffer A); and (b) a container holding components comprising the components of (a) and organic solvent (Buffer B). Optionally, the reaction product of (salt of) secondary or tertiary amine with organic or inorganic acid is selected from an acetate salt of an amine selected from the group consisting of triethylamine, diallylamine, diisopropylamine, N,N-dimethyl-N-cyclohexylamine, N,N-dimethyl-N-isopropylamine, and N,N-dimethyl-N-butylamine; optionally the reaction product of secondary or tertiary amine with organic or inorganic acid is present in the water at a concentration of 1-200 mM; optionally the reaction product of secondary or tertiary amine with organic or inorganic acid is present in the water at a concentration of about 5 mM; optionally the organic solvent is selected from methanol and acetonitrile; optionally the water is HPLC grade water; optionally the kit further includes a (i.e., one or more) chromatography column; optionally the chromatography column is a reverse phase chromatography column; optionally the reverse phase chromatography column is a C18 reverse phase chromatography column; optionally the C18 reverse phase column has a pore size of at least 120 Å; optionally the column has a size of 0.3 mm to 4.6 mm in inner diameter and from 10 mm to 250 mm in length; optionally the kit further includes instructions for preparing a chromatography elution gradient from Buffer A and Buffer B.

The above disclosure generally describes the present invention. A more complete understanding can be obtained by reference to the following specific examples, which are provided herein for purposes of illustration only and are not intended to limit the scope of the invention.

### EXAMPLES

### Example 1

Separation and Identification of Oligonucleotide Fragments That Differ by a Single Nucleotide Using HPLC

This example describes the separation and identification of short DNA fragments by liquid chromatography. The detection of DNA fragments is by UV absorbance and the identification is by retention time on the column compared to standards.

The chromatography system is from Varian (Walnut Creek, Calif.) and is a ProStar Helix System (catalog # Helixsys01) that is composed of two pumps, a column oven, a UV detector, a degasser, a mixer and an autoinjector. The column is a Varian Microsorb MV (catalog number R0086203F5), C18 packing with 5 uM particle size, with 300 Angstroms pore size, 4.6 mm×50 mm. The column was run at 30° C. to 40° C. with a gradient of acetonitrile in 100 mM triethylamine acetate (TEAA) and 0.1 mM EDTA. The type of gradient is described in the text.

The following genotyping fragments, each containing a specific Single Nucleotide Polymorphism were tested and successfully separated.

| | | |
|---|---|---|
| 4-merA: | 5′-ACGA-3′ | |
| 6-merA: | 5′-ACGATG-3′ | |
| 8-merA: | 5′-ACGACGCA-3′ | |
| 8-merB: | 5′-ATGACGCA-3′ | |
| 8-merC: | 5′-ACGATGCA-3′ | |
| 10-merA: | 5′-GAATATCCAT-3′ | (SEQ ID NO.1) |
| 10-merB: | 5′-GAATATCCAC-3′ | (SEQ ID NO.2) |
| 10-merC: | 5′-GAACATCCAT-3′ | (SEQ ID NO.3) |

The polymorphisms in the 8-mers and 10-mers are underlined. The 8-mers B and C differ from 8-mer A by only a single base. The 10-mers B and C differ from 10-mer A by only a single base.

The following HPLC method was used to separate the fragments on the column: Buffer A is 100 mM TEAA with 0.1 mM EDTA, Buffer B is 100 mM TEAA with 0.1 mM EDTA and 25% (V/V) acetonitrile, 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column was run at 40° C. by adjusting the column oven to 40° C. The flow rate was 1.5 ml per minute. The injection volume was 10 microliters and 200 nanogram of fragment was injected per 10 microliter volume. Different combinations of the 4-mer, 6-mer, 8-mer and 10-mer were injected to determine the chromatographic behavior.

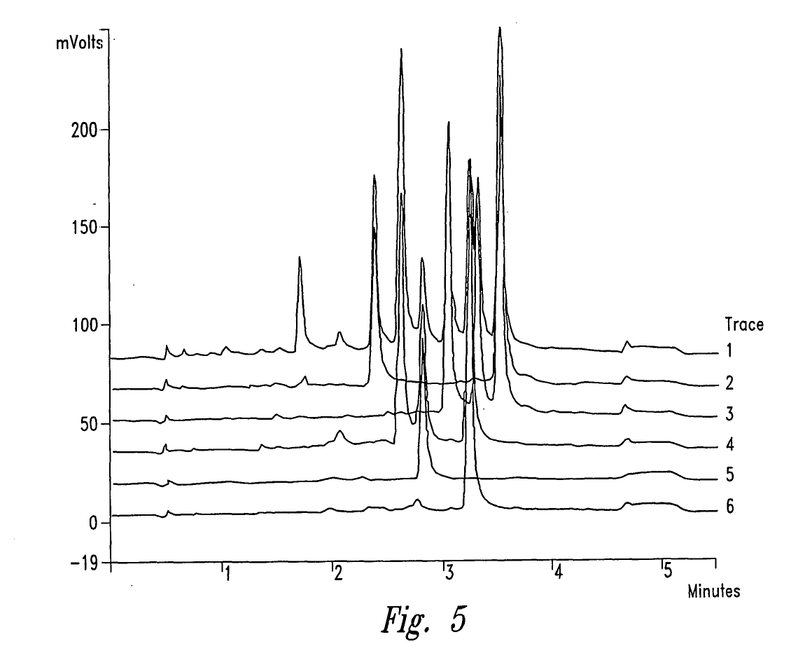

The first result is shown in FIG. 5. In Trace 1 in FIG. 5, all 8 fragments composed of the 4-mer, 6-mer, 8-mer and O-mer were separated. All three 8-mers and all three 10-mers were separated even though they differed by only a single base. The fragments are single-stranded. The order of elution in Trace 1 is (from left to right): 4-mer, 6-mer, 8-merB, 8-merA, 10-merA, 8-merC, 10-merB, 10-merC. In Trace 2, the 6-mer and 10-merC were coinjected and the elution times of the 6-mer and 10-merC were the same as seen in Trace 1. In Trace 3, the three 10-mers were co-injected and separated. The elution times of the three 10-mers were the same as seen in Trace 1. In Trace 4, the three 8-mers were co-injected and separated. The elution times of the three 8-mers were the same as seen in Trace 1. Trace 5 shows a single peak of 8-merA and Trace 6 shows a single Trace of 8-merB. Genotypes can be directly inferred from the retention times during the chromatography, even from fragments that differ by only a single base.

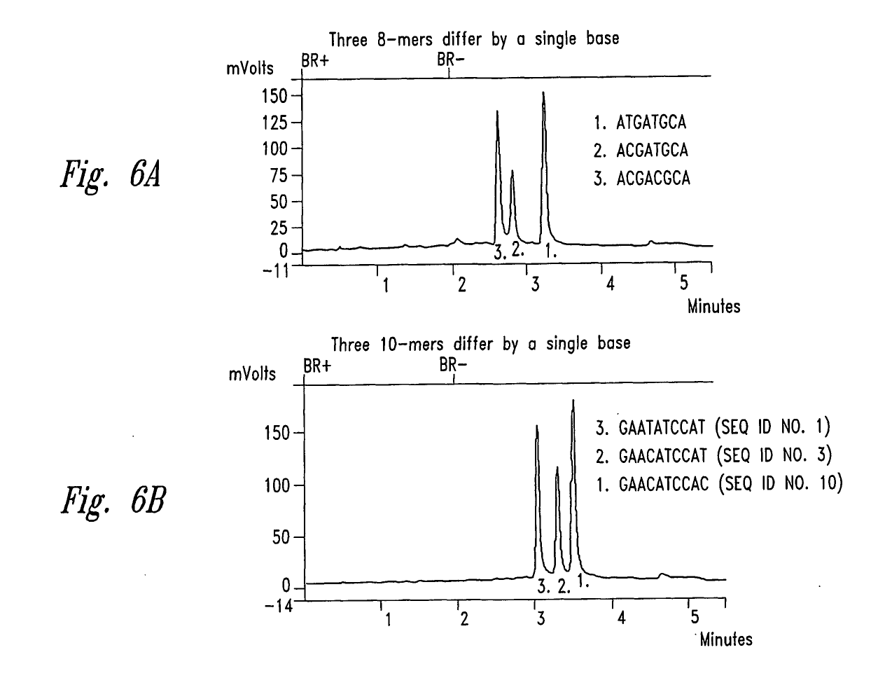

FIG. 6 shows HPLC fractionation and detection of three 8-mers (FIG. 6A) and three 10-mers (FIG. 6B). In panel A, the “T” allele at position 2 of the 1^{st} 8-mer is discriminated from the “C” allele at position 2 of the 2^{nd} 8-mer and the “T” allele at position 5 of the 2^{nd} 8-mer from the “C” allele at position 5 of the 3^{rd} 8-mer. In panel B, the “T” allele at position 4 of the 3^{rd} 1-mer is discriminated from the “C” allele at position 4 of the 2^{nd} 10-mer and the “C” allele at position 10 of the 2^{nd} 10-mer from the “T” allele at position 10 of the 1^{st} 10-mer. Genotypes can be directly inferred from the retention times during the chromatography, even from fragments that differ by only a single base.

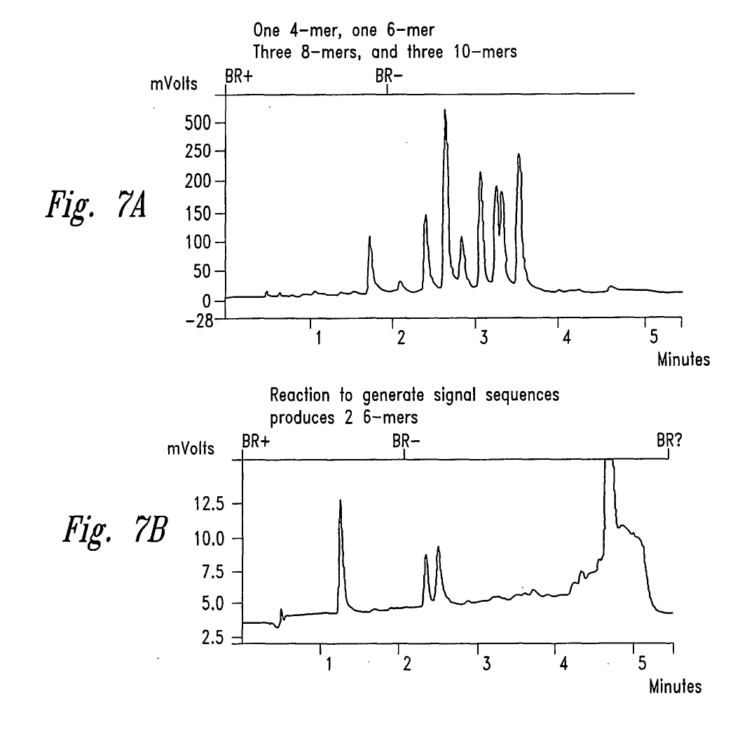

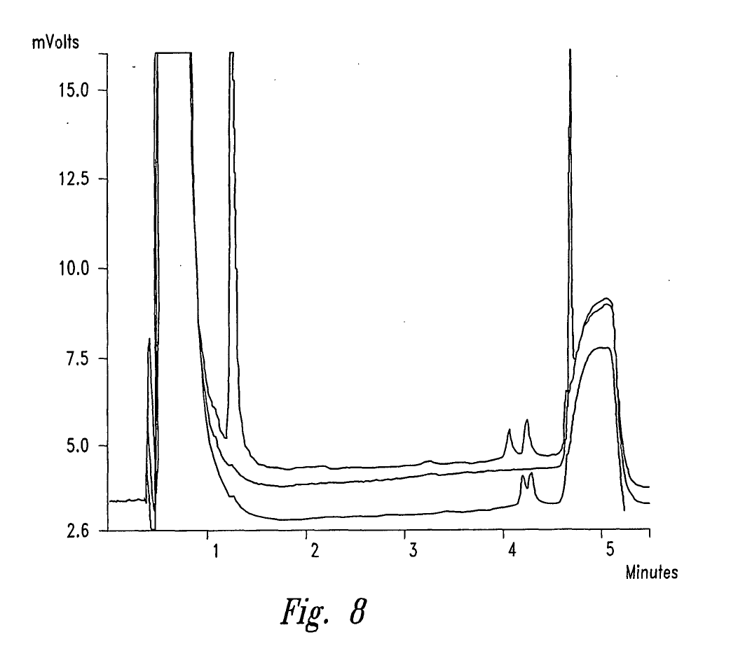

In FIG. 7, in Panel A, one 4-mer (4-merA), one 6-mer (6-merA), three 8-mers (8-merA, 8-merB and 8-merC) and three 10-mers (10-merA, 10-merB, and 10-merC) are separated, and in Panel B, two 6-mers are shown eluting between 2 and 3 minutes. The 6-mers were generated by double Fok I digestion of a 41-mer which contained the forward and reverse Fok I recognition site which was separated by 6 nucleotides.

### Example 2

### Amplification, Cutting, and Detection of a Genotyping Fragment Using the Fok I Restriction Endonuclease

The following example describes the amplification of a specific sequence from the lambda genome in which the primers contain the Fok I recognition sequence (both the forward and reverse primers contain this sequence). The resulting amplicon contains a “double-Fok I” cutting site which liberates a small oligonucleotide fragment, which is then subjected to a chromatography step and identified by UV absorbance.

Two sets of primers were designed to generate two different amplicons from two different regions of the lambda genome. The Fok I recognition sequences of the primers are in bold face.

(SEQ ID NO.4)

RE5P01F: 5′- GAAGTGATGGGGATGCGGAAAGAG-3′

(SEQ ID NO.5)

RE5P02R: 5′- GTAAGCCACAGGATGAGGAACGGG-3′

(SEQ ID NO.6)

RE5P03F: 5′-AAAGCTGGCAGGATGACCGGCAGA -3′

(SEQ ID NO.7)

RE5P04R: 5′-AGCGTCTGTTGGATGTCGTGGCGG -3′

Where RE5P01F and RE5P02R are primer set one and RE5P03F and RE5P₀₄R are primer set 2. All oligonucleotides were synthesized by Midland Reagent CO. of Midland Tex.

The templates for primer set one and two are as follows:

For primer set one:

(SEQ ID NO.8)

5′-GAAGTGATGGCAGAGCGGAAAGAGCATTATTCAGCGCCCGTTCCTGA
CCGTGTGGCTTAC-3′

For primer set two:

(SEQ ID NO.9)

5′-GAAAGCTGGCTGATTGACCGGCAGATTATTATGGGCCGCCACGACGA
TGAACAGACGCTG-3′

The following PCR reaction mixture was used in 25 μl volumes: The 25 μl PCR reactions were composed of 25 ng genomic DNA, 0.5 μM forward and reverse primers, 10 mM Tris pH 8.3, 50 mM KCl, 1.5 mM MgCl₂, 200 μM each dNTP, 4 Units Taq DNA Polymerase (Boehringer Mannheim, Indianapolis, Ind.), and 880 ng TaqStart Antibody (Clontech, Palo Alto, Calif.). Thermocycling conditions were as follows: 94° C. for 5 minutes initial denaturation; 45 cycles of 94° C. for 30 seconds, 60° C. for 30 seconds, 72° C. for 1 minute; final extension at 72° C. for 5 minutes. A MJ Research 9600 thermocycler (MJ Research, Watertown, Mass.) was used for all PCR reactions.

After the thermocycling was complete, 5 microliters of NEB buffer-4 (New England Biolabs, Beverly Mass.) was added, followed by the addition of 5 microliters of Fok I enzyme (New England Biolabs, Beverly Mass.) (20 units), giving final concentrations of 50 mM potassium acetate, 20 mM Tris acetate, 10 mM Mg acetate, 1 mM DTT, pH 7.9. The reaction was carried out at 37° C. for 30 minutes. The reaction was injected directly without any further purification.

The following HPLC method was used to separate the fragments on the column: Buffer A is 100 mM TEAA with 0.1 mM EDTA, Buffer B is 100 mM TEAA with 0.1 mM EDTA and 25% (V/V) acetonitrile, 0-3 minutes there is a gradient of 20% B to 25% B, at 3.01 minutes to 4 minutes, there is a ramp to 45% B, at 4.01 to 4.5 minutes there is a ramp to 95% B, at 4.51 minutes there is 1 minutes hold at 20% B to re-equilibrate the column. The column was run at 40° C. by adjusting the column oven to 40° C. The flow rate was 1.5 ml per minute. The injection volume was 10 to 30 microliters.

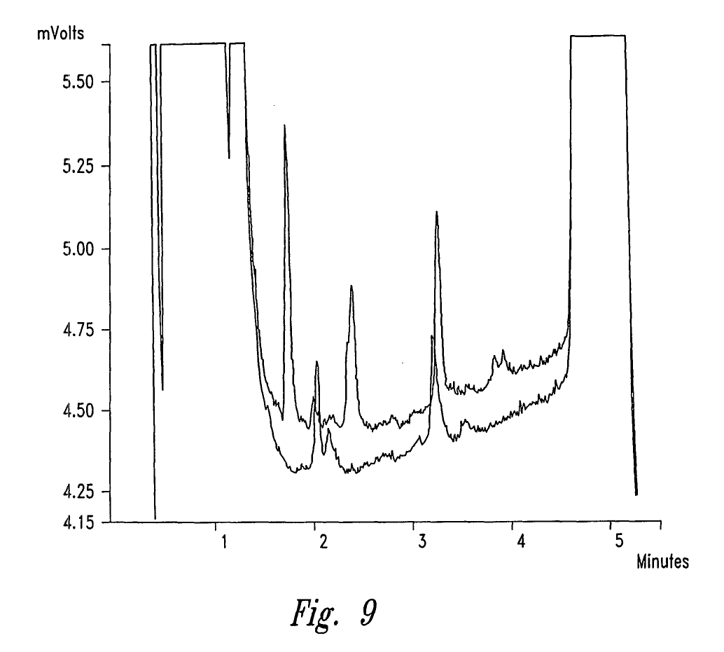

In FIG. 8, the controls for the chromatograms are shown. In Trace 1, the no-template control is shown in which the unincorporated primers (primer set 1) can be seen eluting after the 4 minute mark. In Trace 3, the “no-template” control is shown in which the unincorporated primers (primer set 2) can be seen eluting after the 4 minute mark. In trace 2, the “plus-template” control is seen prior to cutting with Fok I. The large amplicon is seen eluting at 4.6 minutes. In Trace 1, a peak is seen at 1.24 minutes which is due to the Fok I buffer (the no-template control was mixed with the Fok I buffer components as a control). The large peak at 0.5 to 1 minute is due to the PCR components. In FIG. 9 is shown the short fragments generated by the Fok I enzyme double digest. 6-mers (5′-TTATTA-3′ and its complement) and 8-mers (5′-CATTATTC-3′ and its complement) were expected. For primer set one, peaks are seen at 2.0 and 3.2 minutes and for primers set two, peaks are seen at 2.4 and 3.3 minutes. Therefore, SNP fragments can be easily generated and detected by the double-Fok I amplification and cutting.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method of performing liquid chromatography comprising
applying two nucleic acid molecules to a liquid chromatography column, where the two nucleic acid molecules have an identical number of nucleotide bases, but have different nucleotide sequences; and
eluting the two nucleic acid molecules from the column with an elution buffer so that the two nucleic acid molecules have different elution times;
the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid.

2. The method of claim 1 wherein the two nucleic acid molecules are composed of identical nucleotides, but the order of the nucleotides in the two nucleic acid molecules is non-identical.

3. The method of claim 1 wherein the two nucleic acid molecules each have the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X.

4. The method of claims 1-3 wherein the nucleic acid molecules have 3 nucleotides.

5. The method of claims 1-3 wherein the nucleic acid molecules have 4 nucleotides.

6. The method of claims 1-3 wherein the nucleic acid molecules have 5 nucleotides.

7. The method of claims 1-3 wherein the nucleic acid molecules have 6 nucleotides.

8. The method of claims 1-3 wherein the nucleic acid molecules have 7 nucleotides.

9. The method of claims 1-3 wherein the nucleic acid molecules have 8 nucleotides.

10. The method of claims 1-3 wherein the nucleic acid molecules have 9 nucleotides.

11. The method of claims 1-3 wherein the nucleic acid molecules have 10 nucleotides.

12. The method of claims 1-3 wherein the liquid chromatography column is a reverse phase chromatography column.

13. The method of claims 1-3 wherein the liquid chromatography column is a C18 reverse phase chromatography column.

14. The method of claims 1-3 wherein the liquid chromatography column is a C18 reverse phase chromatography column having a pore size of at least 120 Å.

15. The method of claims 1-3 wherein the liquid chromatography column is a C18 reverse phase chromatography column having a pore size of at least 120 Å and a particle size of the solid support within the column is 2 microns to 10 microns.

16. The method of claims 1-3 wherein the liquid chromatography column is a C18 reverse phase chromatography column containing monomeric silica.

17. The method of claims 1-3 wherein the liquid chromatography column is a C18 reverse phase chromatography column having 10-15% carbon load.

18. The method of claims 1-3 wherein the liquid chromatography column is maintained at a temperature between 20° C.-80° C. during at least part of the time the nucleic acid molecules are eluting through the column.

19. The method of claims 1-3 wherein the liquid chromatography column is maintained at a temperature between 30° C.-70° C. during at least part of the time the nucleic acid molecules are eluting through the column.

20. The method of claim 1 wherein Buffer A comprises water and an ammonium salt of a secondary or tertiary amine complexed with an organic or inorganic acid.

21. The method of claim 20 wherein Buffer A has a pH ranging from 5-9.

22. The method of claim 20 wherein Buffer A has a pH ranging from 6-8.

23. The method of claim 20 wherein the ammonium salt is present in Buffer A at a concentration of 1-100 mM.

24. The method of claim 20 wherein the ammonium salt is present in Buffer A at a concentration of 5-50 mM.

25. The method of claim 20 wherein the ammonium salt is of a secondary amine of the formula R₂NH and R at each occurrence is independently selected from C₁-C₁₀hydrocarbon groups.

26. The method of claim 25 wherein the hydrocarbon is an alkyl or cycloalkyl group.

27. The method of claim 20 wherein the ammonium salt is of a secondary amine of the formula R₂NH and R at each occurrence is independently selected from C₁-C₆hydrocarbon groups.

28. The method of claim 27 wherein the hydrocarbon is selected from alkyl and cycloalkyl groups.

29. The method of claim 20 wherein the ammonium salt is of a secondary amine of the formula R₂NH, and R at each occurrence is independently selected from C₅-C₈hydrocarbon groups.

30. The method of claim 29 wherein the hydrocarbon is selected from alkyl and cycloalkyl groups.

31. The method of claim 20 wherein the amine component of the ammonium salt is a secondary amine selected from diallylamine and diisopropylamine.

32. The method of claim 20 wherein the ammonium salt is of a tertiary amine of the formula R₃N and R at each occurrence is independently selected from C₁-C₁₀hydrocarbon groups.

33. The method of claim 32 wherein the hydrocarbon is selected from alkyl and cycloalkyl groups.

34. The method of claim 20 wherein the ammonium salt is of a tertiary amine of the formula R₃N and R at each occurrence is independently selected from C₁-C₁₀hydrocarbon groups.

35. The method of claim 34 wherein the hydrocarbon is selected from alkyl and cycloalkyl groups.

36. The method of claim 20 wherein the ammonium salt is of a tertiary amine of the formula R₃N and R at each occurrence is independently selected from C₁-C₁₀hydrocarbon groups.

37. The method of claim 36 wherein the hydrocarbon is selected from alkyl and cycloalkyl groups.

38. The method of claim 20 wherein the amine component of the ammonium salt is selected from triethylamine, N,N-dimethyl-N-cyclohexylamine, N,N-dimethyl-N-isopropylamine, and N,N-dimethyl-N-butylamine.

39. The method of claim 20-38 wherein the secondary or tertiary amine is complexed with an organic acid.

40. The method of claim 20-38 wherein the secondary or tertiary amine is complexed with an organic acid, and the organic acid is selected from acetic acid, propionic acid, and halogenated versions thereof.

41. The method of claim 20-38 wherein the secondary or tertiary amine is complexed with an organic acid, and the organic acid is acetic acid.

42. The method of claim 20-38 wherein the secondary or tertiary amine is complexed with an organic acid, and the organic acid is formic acid.

43. The method of claim 20-38 wherein the secondary or tertiary amine is complexed with an organic acid, and the organic acid is an acid form of carbonate.

44. The method of claim 20-38 wherein the secondary or tertiary amine is completed with an organic acid, and the organic acid is an acid form of bicarbonate.

45. The method of claim 20-38 wherein the secondary or tertiary amine is complexed with an inorganic acid, and the inorganic acid is hydrochloric acid.

46. The method of claim 1 wherein Buffer B comprises organic solvent and Buffer A.

47. The method of claim 46 wherein Buffer B has a pH ranging from 5-9.

48. The method of claim 46 wherein Buffer A has a pH ranging from 6-8.

49. The method of claim 46 wherein the ammonium salt is present in Buffer A at a concentration of 1-100 mM.

50. The method of claim 46 wherein the ammonium salt is present in Buffer A at a concentration of 5-50 mM.

51. The method of claim 46 wherein the organic solvent comprises acetonitrile.

52. The method of claim 46 wherein the organic solvent comprises methanol.

53. The method of claims 46-52 wherein Buffer B comprises organic solvent and Buffer A in a organic solvent:Buffer A volume ratio of 25-75:75-25.

54. A method of performing liquid chromatography comprising
applying two nucleic acid molecules to a liquid chromatography column, where the two nucleic acid molecules have an identical number of nucleotide bases within the range of 2-10, but have different nucleotide sequences, and the liquid chromatography column is a reverse phase chromatography column; and
eluting the two nucleic acid molecules from the column with an elution buffer so that the two nucleic acid molecules have different elution times;
the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid.

55. The method of claim 54 wherein the two nucleic acid molecules are composed of identical nucleotides, but the order of the nucleotides in the two nucleic acid molecules is non-identical.

56. The method of claim 54 wherein the two nucleic acid molecules each have the sequence 5′-n-X-m-3′, where n and m each represent a sequence of from 0-9 nucleotides, and X represents a single nucleotide, and the two nucleic acid molecules each have the same sequences n and m, but differ in the identify of the nucleotide at location X.

57. The method of claims 54-56 wherein the nucleic acid molecules have 3 nucleotides.

58. The method of claims 54-56 wherein the nucleic acid molecules have 4 nucleotides.

59. The method of claims 54-56 wherein the nucleic acid molecules have 5 nucleotides.

60. The method of claims 54-56 wherein the nucleic acid molecules have 6 nucleotides.

61. The method of claims 54-56 wherein the nucleic acid molecules have 7 nucleotides.

62. The method of claims 54-56 wherein the nucleic acid molecules have 8 nucleotides.

63. The method of claims 54-56 wherein the nucleic acid molecules have 9 nucleotides.

64. The method of claims 54-56 wherein the nucleic acid molecules have 10 nucleotides.

65. The method of claims 54-56 wherein the liquid chromatography column is a C18 reverse phase chromatography column.

66. The method of claims 54-56 wherein the liquid chromatography column is a C18 reverse phase chromatography column having a pore size of at least 120 Å.

67. The method of claims 54-56 wherein the liquid chromatography column is a C18 reverse phase chromatography column having a pore size of at least 120 Å and a particle size of the solid support within the column is 2 microns to 10 microns.

68. The method of claims 54-56 wherein the liquid chromatography column is a C18 reverse phase chromatography column containing monomeric silica.

69. The method of claims 54-56 wherein the liquid chromatography column is a C18 reverse phase chromatography column having 10-15% carbon load.

70. The method of claims 54-56 wherein the liquid chromatography column is maintained at a temperature between 20° C.-80° C. during at least part of the time the nucleic acid molecules are eluting through the column.

71. The method of claims 54-56 wherein the liquid chromatography column is maintained at a temperature between 30° C.-70° C. during at least part of the time the nucleic acid molecules are eluting through the column.

72. The method of claim 54 wherein Buffer A comprises water and an ammonium salt of a secondary or tertiary amine complexed with an organic or inorganic acid.

73. The method of claim 72 wherein Buffer A has a pH ranging from 5-9.

74. The method of claim 72 wherein Buffer A has a pH ranging from 6-8.

75. The method of claim 72 wherein the ammonium salt is present in Buffer A at a concentration of 1-100 mM.

76. The method of claim 72 wherein the ammonium salt is present in Buffer A at a concentration of 5-50 mM.

77. The method of claim 72 wherein the ammonium salt is of a secondary amine of the formula R₂NH and R at each occurrence is independently selected from C₁-C₁₀hydrocarbon groups.

78. The method of claim 77 wherein the hydrocarbon is an alkyl or cycloalkyl group.

79. The method of claim 72 wherein the ammonium salt is of a secondary amine of the formula R₂NH and R at each occurrence is independently selected from C₁-C₆hydrocarbon groups.

80. The method of claim 79 wherein the hydrocarbon is selected from alkyl and cycloalkyl groups.

81. The method of claim 72 wherein the ammonium salt is of a secondary amine of the formula R₂NH, and R at each occurrence is independently selected from C₅-C₈hydrocarbon groups.

82. The method of claim 81 wherein the hydrocarbon is selected from alkyl and cycloalkyl groups.

83. The method of claim 72 wherein the amine component of the ammonium salt is a secondary amine selected from diallylamine and diisopropylamine.

84. The method of claim 72 wherein the ammonium salt is of a tertiary amine of the formula R₃N and R at each occurrence is independently selected from C₁-C₁₀hydrocarbon groups.

85. The method of claim 84 wherein the hydrocarbon is selected from alkyl and cycloalkyl groups.

86. The method of claim 72 wherein the ammonium salt is of a tertiary amine of the formula R₃N and R at each occurrence is independently selected from C₁-C₁₀hydrocarbon groups.

87. The method of claim 86 wherein the hydrocarbon is selected from alkyl and cycloalkyl groups.

88. The method of claim 72 wherein the ammonium salt is of a tertiary amine of the formula R₃N and R at each occurrence is independently selected from C₁-C₁₀hydrocarbon groups.

89. The method of claim 88 wherein the hydrocarbon is selected from alkyl and cycloalkyl groups.

90. The method of claim 72 wherein the amine component of the ammonium salt is selected from triethylamine, N,N-dimethyl-N-cyclohexylamine, N,N-dimethyl-N-isopropylamine, and N,N-dimethyl-N-butylamine.

91. The method of claim 72-90 wherein the secondary or tertiary amine is complexed with an organic acid.

92. The method of claim 72-90 wherein the secondary or tertiary amine is complexed with an organic acid, and the organic acid is selected from acetic acid, propionic acid, and halogenated versions thereof.

93. The method of claim 72-90 wherein the secondary or tertiary amine is complexed with an organic acid, and the organic acid is acetic acid.

94. The method of claim 72-90 wherein the secondary or tertiary amine is complexed with an organic acid, and the organic acid is formic acid.

95. The method of claim 72-90 wherein the secondary or tertiary amine is complexed with an organic acid, and the organic acid is an acid form of carbonate.

96. The method of claim 72-90 wherein the secondary or tertiary amine is complexed with an organic acid, and the organic acid is an acid form of bicarbonate.

97. The method of claim 72-90 wherein the secondary or tertiary amine is complexed with an inorganic acid, and the inorganic acid is hydrochloric acid.

98. The method of claim 54 wherein Buffer B comprises organic solvent and Buffer A.

99. The method of claim 98 wherein Buffer B has a pH ranging from 5-9.

100. The method of claim 98 wherein Buffer A has a pH ranging from 6-8.

101. The method of claim 98 wherein the ammonium salt is present in Buffer A at a concentration of 1-100 mM.

102. The method of claim 98 wherein the ammonium salt is present in Buffer A at a concentration of 5-50 mM.

103. The method of claim 98 wherein the organic solvent comprises acetonitrile.

104. The method of claim 98 wherein the organic solvent comprises methanol.

105. The method of claims 98-105 wherein Buffer B comprises organic solvent and Buffer A in a organic solvent:Buffer A volume ratio of 25-75:75-25.

106. A method of performing liquid chromatography comprising
applying two nucleic acid molecules to a liquid chromatography column, where the two nucleic acid molecules have an identical number of nucleotide bases, but have different nucleotide sequences; and
eluting the two nucleic acid molecules from the column with an elution buffer so that the two nucleic acid molecules have different elution times;
the elution buffer being formed from Buffer A and Buffer B, where Buffer A comprises water and an ammonium salt that is formed from a secondary or tertiary amine complexed with an organic or inorganic acid; and Buffer B comprises water, organic solvent, and an ammonium salt that is formed from a secondary or tertiary amine complexed with an organic or inorganic acid;
where elution buffer of incrementally increasing organic solvent concentration is applied to the column.

107. The method of claim 106 wherein the two nucleic acid molecules are composed of identical nucleotides, but the order of the nucleotides in the two nucleic acid molecules is non-identical.

108. The method of claim 107 wherein the two nucleic acid molecules each have the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X.

109. The method of claim 108 wherein the two nucleic acid molecules each have 2-10 nucleotides.

110. A method of performing liquid chromatography and mass spectrometric analysis comprising
applying a plurality of pairs of nucleic acid molecules to a liquid chromatography column, where each pair of nucleic acid molecules is formed from two nucleic acid molecules that have an identical number of nucleotide bases, but have different nucleotide sequences; and
eluting the plurality of pairs of nucleic acid molecules from the column with an elution buffer so that the two nucleic acid molecules that form each pair have different elution times;
characterizing each nucleic acid molecule by mass spectroscopy;
the elution buffer being formed from Buffer A and Buffer B, where elution buffer of incrementally increasing organic solvent concentration is applied to the column, where the elution buffer comprises an ammonium salt and the ammonium salt comprises a secondary or tertiary amine complexed with an organic or inorganic acid.

111. The method of claim 110 wherein each member of a pair of two nucleic acid molecules is composed of identical nucleotides, but the order of the nucleotides in the two nucleic acid molecules is non-identical.

112. The method of claim 110 wherein each member of a pair of two nucleic acid molecules has the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X.

113. The method of claims 110-112 wherein the liquid chromatography column is a reverse phase chromatography column.

114. The method of claims 110-112 wherein the liquid chromatography column is a C18 reverse phase chromatography column.

115. The method of claims 110-112 wherein the liquid chromatography column is a C18 reverse phase chromatography column having a pore size of at least 120 Å.

116. The method of claims 110-112 wherein the liquid chromatography column is a C18 reverse phase chromatography column having a pore size of at least 120 Å and a particle size of the solid support within the column is 2 microns to 10 microns.

117. The method of claims 110-112 wherein the liquid chromatography column is a C18 reverse phase chromatography column containing monomeric silica.

118. The method of claims 110-112 wherein the liquid chromatography column is a C18 reverse phase chromatography column having 10-15% carbon load.

119. The method of claims 110-112 wherein the liquid chromatography column is maintained at a temperature between 20° C.-80° C. during at least part of the time the nucleic acid molecules are eluting through the column.

120. The method of claims 110-112 wherein the liquid chromatography column is maintained at a temperature between 30° C.-70° C. during at least part of the time the nucleic acid molecules are eluting through the column.

121. A composition referred to as Buffer B comprising water, the reaction product of secondary or tertiary amine with organic or inorganic acid, and organic solvent.

122. The composition of claim 121 wherein the reaction product of secondary or tertiary amine with organic or inorganic acid is selected from an acetate salt of an amine selected from the group consisting of triethylamine, diallylamine, diisopropylamine, N,N-dimethyl-N-cyclohexylamine, N,N-dimethyl-N-isopropylamine, and N,N-dimethyl-N-butylamine.

123. The composition of claim 121 having a pH of 6-8.

124. The composition of claim 121 wherein the organic solvent is selected from methanol and acetonitrile.

125. The composition of claim 121 having a water:organic solvent ratio of 95-25:5-75.

126. The composition of claim 121 wherein the reaction product is present in Buffer B at a concentration of 1-100 mM.

127. A composition comprising water, the reaction product of secondary or tertiary amine with organic or inorganic acid, organic solvent, and two nucleic acid molecules, where the two nucleic acid molecules have an identical number of nucleotide bases, but have different nucleotide sequences.

128. The composition of claim 127 wherein the two nucleic acid molecules are composed of identical nucleotides, but the order of the nucleotides in the two nucleic acid molecules is non-identical.

129. The composition of claim 127 wherein the two nucleic acid molecules each have the sequence 5′-n-X-m-3′, where n and m each represent a sequence of from 0-10 nucleotides, and X represents a single nucleotide, and the two nucleic acid molecules each have the same sequences n and m, but differ in the identify of the nucleotide at location X.

130. The composition of claim 127-129 wherein the two nucleic acid molecules have 4-10 nucleotides.

131. The composition of claim 127 wherein the reaction product of secondary or tertiary amine with organic or inorganic acid is selected from an acetate salt of an amine selected from the group consisting of triethylamine, diallylamine, diisopropylamine, N,N-dimethyl-N-cyclohexylamine, N,N-dimethyl-N-isopropylamine, and N,N-dimethyl-N-butylamine.

132. The composition of claim 127 having a pH of 6-8.

133. The composition of claim 127 wherein the organic solvent is selected from methanol and acetonitrile.

134. The composition of claim 127 having a water:organic solvent ratio of 95-25:5-75.

135. The composition of claim 127 comprising four nucleic acid molecules, the four nucleic acid molecules being two pairs of nucleic acid molecules, each pair of nucleic acid molecules being formed from two nucleic acid molecules that have an identical number of nucleotide bases, but have different nucleotide sequences.

136. A kit for chromatographic analysis comprising
(a) a container holding components comprising water and the reaction product of secondary or tertiary amine with organic or inorganic acid (Buffer A); and
(b) a container holding components comprising the components of (a) and organic solvent (Buffer B).

137. The kit of claim 136 wherein the reaction product of secondary or tertiary amine with organic or inorganic acid is selected from an acetate salt of an amine selected from the group consisting of triethylamine, diallylamine, diisopropylamine, N,N-dimethyl-N-cyclohexylamine, N,N-dimethyl-N-isopropylamine, and N,N-dimethyl-N-butylamine.

138. The kit of claim 137 wherein the reaction product of secondary or tertiary amine with organic or inorganic acid is present in the water at a concentration of 1-200 mM.

139. The kit of claim 137 wherein the reaction product of secondary or tertiary amine with organic or inorganic acid is present in the water at a concentration of about 5 mM.

140. The kit of claim 137 wherein the organic solvent is selected from methanol and acetonitrile.

141. The kit of claim 137 wherein the water is HPLC grade water.

142. The kit of claim 137 further comprising a chromatography column.

143. The kit of claim 142 wherein the chromatography column is a reverse phase chromatography column.

144. The kit of claim 142 wherein the reverse phase chromatography column is a C18 reverse phase chromatography column.

145. The kit of claim 144 wherein the C18 reverse phase column has a pore size of at least 120 Å.

146. The kit of claim 142 wherein the column has a size of 0.3 mm to 4.6 mm in inner diameter and from 10 mm to 250 mm in length.

147. The kit of claim 142 further comprising instructions for preparing a chromatography elution gradient from Buffer A and Buffer B.

148. A method for identifying one or more nucleotide(s) at a defined location in a double-stranded target nucleic acid, comprising
(a) forming a mixture of the target nucleic acid, a first oligonucleotide primer (ODNP) and a second ODNP,
wherein each of the first and the second ODNPs comprises a 5′ end and a 3′ end, wherein a first portion of each ODNP at the 5′ end and a second portion of each ODNP at the 3′ end are at least substantially complementary to a first portion and a second portion of the target nucleic acid, wherein 4-8 nucleotides between the first portion and the second portion of the ODNP comprise a recognition sequence for a restriction endonucleotide (RE) that cleaves outside its recognition site,
wherein each ODNP is complementary to an opposite strand of the target nucleic acid, the first and the second ODNPs are at least partially complementary to two non-contiguous regions of the target nucleic acid, and the defined position is between the two non-contiguous regions;
(b) amplifying the target nucleic acid using the first and the second ODNPs;
(c) digesting the amplification product of step (b) with the restriction endonuclease(s) that recognize the recognition sites formed from the first and the second ODNPs; and
(d) characterizing a short digestion product of step (c) with liquid chromatography to identify the one or more nucleotides at the defined position.

149. A method for identifying one or more nucleotide bases at a defined position of a polynucleotide, comprising:
(1) combining the polynucleotide with a first oligonucleotide primer (ODNP), the first ODNP comprising
(a) a nucleotide base sequence that is a recognition sequence for a restriction endonuclease, the restriction endonuclease having a cleavage site outside the recognition sequence,
(b) a nucleotide base sequence that enables the first ODNP to anneal to the polynucleotide at a location 3′ to the defined position, and
(c) a 3′ end that may be extended by a polymerase under primer extension conditions;
(2) extending the first ODNP to incorporate the complement of the base(s) at the defined position, and additional bases 5′ to the defined position, so as to provide an extended first ODNP;
(3) combining a second ODNP with the extended first ODNP and/or the complement of the polynucleotide, the second ODNP comprising
(a) a nucleotide base sequence that is a recognition sequence for a restriction endonuclease, the restriction endonuclease having a cleavage site outside the recognition sequence,
(b) a nucleotide base sequence that enables the second ODNP to anneal to the complement of the polynucleotide at a position 3′ to the complement of the defined position, and
(c) a 3′ end that may be extended by a polymerase under primer extension conditions;
(4) extending the second ODNP to incorporate the bases of the defined position and additional bases 5′ to the defined position, so as to provide an extended second ODNP
(5) combining the extended first and the extended second ODNPs, optionally with the amplification product thereof as formed from additional first and second ODNPs acting on the extended first and the extended second ODNPs as template nucleic acids for primer extension, with restriction enzyme(s) that recognize the recognition sequence(s), under conditions where the restriction enzyme(s) cleave a double-stranded fragment comprising the defined location and the complement thereof, so as to provide the bases to be identified in a short nucleic acid molecule;
(6) characterizing the short nucleic acid molecule by liquid chromatography, so as to identify the one or more nucleotide bases at the defined position.

150. The method of claims 148-149 wherein the first portion of each ODNP at the 5′ end and the second portion of each ODNP at the 3′ end are exactly complementary to the first portion and the second portion of the target nucleic acid or polynucleotide.

151. The method of claims 148-150 wherein the first portion of each ODNP is at least 8 nucleotides in length.

152. The method of claims 148-151 wherein the second portion of each ODNP is at least 3 nucleotides in length.

153. The method of claims 148-151 wherein the second portion of each ODNP is at most 20 nucleotides in length.

154. The method of claims 148-153 wherein the distance between the first and the second portions in the target nucleic acid or the polynucleotide is up to 10 nucleotides in length.

155. The method of claims 148-153 wherein the distance between the first and the second portions in the target nucleic acid or the polynucleotide is 4-8 nucleotides in length.

156. The method of claims 148-153 wherein the recognition sequences in the first and the second ODNPs are the same.

157. The method of claims 148-153 wherein the recognition sequences in the first and the second ODNPs are different.

158. The method of claims 148-157 wherein the restriction enzymes (RE(s)) that recognize the recognition sequences in the first and the second ODNPs are Type IIS RE(s).

159. The method of claims 148-157 wherein the RE(s) is Bpm I.

160. The method of claims 148-159 wherein the distance of the two non-contiguous regions in the target nucleic acid is 1 to 10 nucleotides in length.

161. The method of claims 148-160 wherein the target nucleic acid or the polynucleotide is genomic DNA or cDNA.

162. The method of claims 148-161 wherein the nucleotide(s) at the defined location is associated with a disease.

163. The method of claims 148-161 wherein the nucleotide(s) at the defined location is associated with drug resistance of a pathogenic microorganism.


SEQUENCE LISTING (10 sequences)

SEQ ID NO: 1 - DNA, 10 bp, Artificial Sequence
  Oligonucleotide sequence containing single nucleotide polymorphism used in separation and identification experiments.
gaatatccat

SEQ ID NO: 2 - DNA, 10 bp, Artificial Sequence
  Oligonucleotide sequence containing single nucleotide polymorphism used in separation and identification experiments.
gaatatccac

SEQ ID NO: 3 - DNA, 10 bp, Artificial Sequence
  Oligonucleotide sequence containing single nucleotide polymorphism used in separation and identification experiments.
gaacatccat

SEQ ID NO: 4 - DNA, 24 bp, Artificial Sequence
  Primer designed to generate different amplicons from different regions of the lambda genome.
gaagtgatggggatgcggaaagag

SEQ ID NO: 5 - DNA, 24 bp, Artificial Sequence
  Primer designed to generate different amplicons from different regions of the lambda genome.
gtaagccacaggatgaggaacggg

SEQ ID NO: 6 - DNA, 24 bp, Artificial Sequence
  Primer designed to generate different amplicons from different regions of the lambda genome.
aaagctggcaggatgaccggcaga

SEQ ID NO: 7 - DNA, 24 bp, Artificial Sequence
  Primer designed to generate different amplicons from different regions of the lambda genome.
agcgtctgttggatgtcgtggcgg

SEQ ID NO: 8 - DNA, 60 bp, Artificial Sequence
  Template for primer set one.
gaagtgatggcagagcggaaagagcattattcagcgcccgttcctgaccgtgtggcttac

SEQ ID NO: 9 - DNA, 60 bp, Artificial Sequence
  Template for primer set two.
gaaagctggctgattgaccggcagattattatgggccgccacgacgatgaacagacgctg

SEQ ID NO: 10 - DNA, 10 bp, Artificial Sequence
  Oligonucleotide sequence containing single nucleotide polymorphism used in separation and identification experiments.
gaacatccac